SEQUENCE LISTING (13 sequences)

SEQ ID NO: 1 - protein, 5 aa, synthetic construct
  VARIANT 4: note = hydrophobic block, Xaa = Any Amino Acid Other Than Proline
VPGXG

SEQ ID NO: 2 - protein, 5 aa, synthetic construct
  REGION 1..5: note = aggregation-enhancing block
IPAVG

SEQ ID NO: 3 - protein, 6 aa, synthetic construct
  REGION 1..6: note = beta sheet formation-inducing block
GAGAGS

SEQ ID NO: 4 - protein, 6 aa, synthetic construct
  REGION 1..6: note = beta sheet formation-inducing block
GAGAGY

SEQ ID NO: 5 - protein, 6 aa, synthetic construct
  REGION 1..6: note = beta sheet formation-inducing block
GAGYGA

SEQ ID NO: 6 - protein, 6 aa, synthetic construct
  REGION 1..6: note = beta sheet formation-inducing block
GAGAGA

SEQ ID NO: 7 - protein, 12 aa, synthetic construct
  REGION 1..12: note = biomineralizing block
VTKHLNQISQSY

SEQ ID NO: 8 - protein, 5 aa, synthetic construct
  REGION 1..5: note = hydrophobic block
VPGVG

SEQ ID NO: 9 - protein, 5 aa, synthetic construct
  REGION 1..5: note = hydrophobic block
VPGIG

SEQ ID NO: 10 - protein, 5 aa, synthetic construct
  REGION 1..5: note = hydrophobic block
VPGEG

SEQ ID NO: 11 - protein, 432 aa, synthetic construct
  REGION 1..432: note = Elastin-like polypeptide EPR011
IPAVGIPAVGIPAVGIPAVGVPGVGVPGVGVPGIGVPGVGVPGVGVPGVGVPGVGVPGIGVPGVGVPGVGVPGVGVPGVGVPGIGVPGVGVPGVGVPGVGVPGVGVPGIGVPGVGVPGVGGAGAGSGAGAGSGAGAGSGAGAGSIPAVGIPAVGIPAVGIPAVGVPGVGVPGVGVPGIGVPGVGVPGVGVPGVGVPGVGVPGIGVPGVGVPGVGVPGVGVPGVGVPGIGVPGVGVPGVGVPGVGVPGVGVPGIGVPGVGVPGVGGAGAGSGAGAGSGAGAGSGAGAGSIPAVGIPAVGIPAVGIPAVGVPGVGVPGVGVPGIGVPGVGVPGVGVPGVGVPGVGVPGIGVPGVGVPGVGVPGVGVPGVGVPGIGVPGVGVPGVGVPGVGVPGVGVPGIGVPGVGVPGVGGAGAGSGAGAGSGAGAGSGAGAGS

SEQ ID NO: 12 - protein, 468 aa, synthetic construct
  REGION 1..468: note = Elastin-like polypeptide EPR018
IPAVGIPAVGIPAVGIPAVGVPGVGVPGVGVPGIGVPGVGVPGVGVPGVGVPGVGVPGIGVPGVGVPGVGVTKHLNQISQSYVPGVGVPGVGVPGIGVPGVGVPGVGVPGVGVPGVGVPGIGVPGVGVPGVGGAGAGSGAGAGSGAGAGSGAGAGSIPAVGIPAVGIPAVGIPAVGVPGVGVPGVGVPGIGVPGVGVPGVGVPGVGVPGVGVPGIGVPGVGVPGVGVTKHLNQISQSYVPGVGVPGVGVPGIGVPGVGVPGVGVPGVGVPGVGVPGIGVPGVGVPGVGGAGAGSGAGAGSGAGAGSGAGAGSIPAVGIPAVGIPAVGIPAVGVPGVGVPGVGVPGIGVPGVGVPGVGVPGVGVPGVGVPGIGVPGVGVPGVGVTKHLNQISQSYVPGVGVPGVGVPGIGVPGVGVPGVGVPGVGVPGVGVPGIGVPGVGVPGVGGAGAGSGAGAGSGAGAGSGAGAGS

SEQ ID NO: 13 - protein, 15 aa, synthetic construct
  REGION 1..15: note = biomineralizing block
DDDEEKFLRRIGRFG

(19) United States
(12) Patent Application Publication
(10) Pub. No.: US 2025/0065011 A1
(43) Pub. Date: Feb. 27, 2025

(54) SYNTHETIC BIOPOLYMERS AND THEIR USE IN COMPOSITIONS FOR TISSUE REPAIR

(71) Applicant: DSM IP ASSETS B.V., Heerlen (NL)

(72) Inventors: Diego LÓPEZ BARREIRO, Echt (NL); Jens Christoph THIES, Echt (NL); Timothy A RINGEISEN, Columbia, MD (US); Inge Jeannette MINTEN, Echt (NL)

(21) Appl. No.: 18/723,269

(22) PCT Filed: Dec. 21, 2022
(86) PCT No.: PCT/EP2022/087378

§ 371 Date: Jun. 23, 2024

Related U.S. Application Data
(60) Provisional application No. 63/293,166, filed on Dec. 23, 2021.

(51) Int. Cl. A61L 27/22; A61L 27/46; A61L 27/56; C07K 14/78

(52) U.S. Cl. CPC A61L 27/227; A61L 27/46; A61L 27/56; C07K 14/78; A61L 2400/06; A61L 2430/02

(57) ABSTRACT

Synthetic biopolymers are disclosed, which can be engineered in terms of both their amino acid sequences and post-synthesis treatments to which they are subjected, to achieve desired gelation characteristics, particularly upon being exposed to physiological temperature (e.g., 37° C.). These synthetic biopolymers are therefore useful for injectable or implantable compositions for tissue repair, such as for bone void filler compositions. Examples of synthetic biopolymers are synthetic elastin-like polypeptides (ELPs) having polypeptide sequences with functional oligopeptide blocks that may include one or more hydrophobic blocks, one or more aggregation-enhancing blocks, and one or more β-sheet formation-inducing blocks. Optionally, one or more biomineralizing blocks may be present, to promote bone repair, growth, and/or strengthening.

### CROSS-REFERENCE TO RELATED APPLICATIONS

This application is an International Application that claims priority to U.S. provisional application No. 63/293,166, filed Dec. 23, 2021, which is incorporated by reference in its entirety.

This application incorporates by reference the contents of a 17,992 byte xml file created on Dec. 20, 2022 and named “00749000063SequenceListing.xml,” which is the sequence listing for this application.

### FIELD OF THE INVENTION

Aspects of the invention relate to compositions for tissue repair, such as bone void filler compositions, comprising a synthetic biopolymer, such as an elastin-like polypeptide (ELP).

### DESCRIPTION OF RELATED ART

Improved products and methods that enhance the body's innate ability to heal musculoskeletal injuries and/or that can otherwise aid in the repair of bone and tissue damage (e.g., resulting from degenerative diseases) are subjects of ongoing research and development. Progress in the area of biomaterials has promoted the development of devices for these purposes, broadly relating to the treatment of bone and soft tissue defects, such as those resulting from trauma or advanced age. These materials have shown to be effective, for example, as tissue (e.g., bone) scaffolds, in spinal applications, and for the delivery of drugs and growth factors. In certain medical (e.g., orthopedic) applications, it can become necessary to provide an implant for augmenting injured or diseased bone, thereby accelerating post-operative healing, stimulating repair, and/or preventing further damage. Depending on the specific application, an appropriate implant can assume a wide variety of forms (e.g., sponges, putties, plugs, rods, dowels, wedges, screws, plates, etc.), structural characteristics (e.g., it can be rigid, flexible, deformable, flowable etc.), shapes, and sizes. In the case of rigid implants such as bone screws, the defect site is typically preconditioned by forming a depression, channel, pre-tapped hole, or other feature for receiving the implant.

On the other hand, non-rigid structural repair materials, such as putties, pastes, and sponges, must be capable of adopting a variety of complex shapes to fit the contours of the repair site, while effectively resisting migration (or washout) over time. These properties are needed to achieve the desired integration between the implant and natural tissue, thereby improving the healing process. In the case of bone repair, for example, robust and sustained contact between the substitute and native tissue is important for promoting bone remodeling and regeneration, resulting from incorporation of the repair material by host bone. With the objective of realizing these characteristics, implantable medical devices known as bone void fillers have been developed and successfully implemented in bridging gaps in bone, stabilizing fractures, and encouraging bone growth. These materials (grafts) resemble natural bone and are favorable for cell ingrowth that is needed to ultimately replace the implant with tissue. Whereas some bone void fillers are self-setting into hard solids (e.g., curable polymethyl methacrylate (PMMA) compositions that form cements), non-setting bone void filler implants generally remain in a deformable state for some time after implantation. This is characteristic of sponge or putty compositions, which may optionally include hard particles. For example, granules of hydroxyapatite mineral, calcium phosphate, bioactive glasses, and/or bone particles may be dispersed within a matrix having a porous structure that holds the granules in place and allows for tissue ingrowth.

Non-setting bone void filler implants are normally provided as dry (e.g., freeze-dried or lyophilized) compositions that can be rehydrated, such as to form an injectable putty or an implantable strip or sponge. Examples of rehydrating fluids are sterile water, whole blood, platelet rich plasma (PRP), and bone marrow aspirate. The rehydration, which typically occurs in the operating room or elsewhere in the vicinity of the end use, alters the original dry composition, to provide a consistency and/or pliability that is suitable for implantation into a bone defect, such as directly by hand or by injection through a syringe. More generally, rehydration results in a more flowable, wetted material, e.g., a putty or paste, which can be conformed to a defect site with sufficient rigidity to remain in place, allowing for subsequent bone growth and material resorption.

These compositions are conventionally based on the use of collagen fibers to provide a suitable matrix for bone cell growth and optionally for the retention of bone-mimicking hard particles as described above, generally referred to as mineral particles. According to one example, a product in the form of dry composition, such as a hydratable sponge, can be prepared by forming an aqueous slurry of natural collagen fibers, soluble collagen, and the particles, spreading this aqueous slurry into a mold (usually a large number of molds) having a desired size and shape, and lyophilizing the shaped material. The collagen fibers entangle with and hold the mineral particles, while providing sufficient structural rigidity of the porous sponge. Examples of this technology are described in U.S. Pat. Nos. 7,824,703 and 7,166,133. One commercially available product is MASTERGRAFT® Putty by Medtronic®, which is provided as a dish-shaped sponge of a lyophilized composition, having about 80 wt-% mineral and about 20 wt-% of Type I bovine collagen. The mineral particles, dispersed throughout the sponge, are about 15 wt-% hydroxyapatite and about 85 wt-% β-tricalcium phosphate, based on the weight of the particles. The dish form provides a reservoir for containing the appropriate amount of hydrating fluid (biocompatible wetting fluid), thereby preventing errors upon hydration, with respect to fluid amount and/or fluid losses.

Fibers of natural collagen are typically 2-20 mm in length, and the individual collagen protein molecules that assemble into these fibers retain the triple helical conformation of their peptide chains, as found in their normal biological state (e.g., in connective tissue). Acid soluble collagen, also a conventional component of bone void filler compositions (e.g., hydratable dry putty or a hydratable dry sponge), does not have this fiber conformation, but serves as a useful agent to improve handling properties upon hydration. Compositions for tissue repair that are based on collagen have proven effective as media for bone growth over a desired time scale, and have gained widespread acceptance by the medical community. Collagen, the most abundant protein in the body, has physical and chemical properties that, overall, have shown to be favorable generally for structural and hemostatic applications in both clinical and diagnostic settings.

Despite these advantages, obtaining bovine collagen with properties suitable for medical applications is a complicated process involving numerous steps. The potential use of enzymes or solvents, as well as the requirement, in some cases, for both soluble and insoluble (fibrous) collagen forms for hydratable bone void filler compositions add to the complexities associated with their manufacture and sustainability. Overall, the efforts currently needed to make collagen products involve considerable expense, time, and potential supply chain issues (e.g., associated with controlled herds). Moreover, collagen products are animal-derived and for this reason they may be undesirable for certain subsets of patients. Achieving the benefits of natural bovine collagen but with a more efficient and environmentally friendly, alternative solution would be transformative in the industry.

### SUMMARY OF THE INVENTION

Aspects of the invention are associated with the discovery of synthetic biopolymers, such as those produced by bacterial fermentation and expression in cell culture, having suitable properties for use in implantable compositions. Such compositions may be more particularly designed for use in implantation in a human or animal body to repair tissue, for example as bone void filler compositions. These compositions advantageously do not rely primarily, or at all, on collagen. Consequently, the processing requirements associated with collagen and their environmental impact, as noted above, can be reduced or eliminated in the preparation of synthetic biopolymers and compositions as described herein. Particular aspects relate to the discovery that synthetic elastin-like polypeptides, having analogous functional oligopeptide blocks to native elastin, are exemplary synthetic biopolymers that can replace insoluble (fibrous) collagen and/or soluble collagen as currently used in tissue repair compositions, including bone void filler compositions. Importantly, the suitability of any synthetic biopolymer (e.g., polypeptide) for this purpose depends on whether it performs in a satisfactory manner relative to collagen, in terms of forming compositions that can be shaped and/or manipulated for manufacturing and medical preparation, while also providing necessary structural and/or mineral retaining characteristics, e.g., as needed to encourage bone growth when implanted in the physiological environment in a patient. That is, certain characteristics may be achieved, analogous to the ability of collagen fibers to entangle and form stable, biocompatible scaffolds. Important qualities of the synthetic biopolymer in this regard include the ability to gel when a solution of the biopolymer is exposed to body temperature.

Particular aspects relate to the discovery that desired properties of a synthetic biopolymer, such as a synthetic elastin-like polypeptide (ELP), can depend on both (i) its primary structure, according to its amino acid sequence, as well as (ii) other structures, such as its secondary structure, which can be influenced by post-synthesis treatment. For example, a synthetic ELP can be engineered or configured to achieve desired gelation characteristics, by virtue of property (1), the functional oligopeptide blocks in its polypeptide sequence, and/or property (2), post-synthesis treatment that influences secondary structure to “predispose” the synthetic ELP to physical cross-linking. In the case of property (2), such treatment can include (i) freeze-drying (lyophilization), (ii) water vapor annealing, (iii) washing with an organic liquid, and/or (iv) thermal exposure, any of which, or any combinations of which, can induce physical cross-linking such as by ß-sheet formation among polypeptide molecules, thereby templating or facilitating a subsequent gelling of a composition prepared from the synthetic ELP, when such composition is implanted. Without being bound by theory, it is believed that property (1) can be engineered to principally influence gelation temperature, whereas a combination of property (1) and property (2) can be engineered to more beneficially influence gelation kinetics (e.g., such that gelation sufficiency or strength occurs over a practical time scale, which may be less than 2 hours, less than 1 hour, less than 30 minutes, or less than 15 minutes). Sufficient gelation or strength may occur over such representative amounts of time, or, in other embodiments, sufficient gelation or strength may occur over a time scale, by virtue of properties (1) and (2) in combination, that is reduced by these representative amounts of time, compared to a baseline time scale for sufficient gelation or strength, resulting from property (1) alone. In the case of property (1), this may be engineered, more particularly, such that gelation, or the onset of gelation, occurs at a desired temperature, such as at physiological temperature or possibly below this temperature. For example, it may be desirable to engineer property (1) to achieve an onset of gelation that occurs at a temperature from about 25° C. to about 37° C., or from about 30° C. to about 37° C. In some cases, property (1), optionally in combination with property (2), can be further engineered to provide a synthetic biopolymer that gels irreversibly. Importantly, property (2) can be tailored to provide or induce a given degree of ß-sheet formation, which, optionally in combination with other phenomena such as entanglement of polypeptide strands (“spaghetti” formation), serves to initiate the physical cross-linking ultimately needed for providing a gelled implant with sufficient structural rigidity for tissue (e.g., bone) repair applications.

Particular embodiments of the invention are directed to a synthetic biopolymer that is engineered or configured to undergo gelation, following heating of a solution of the synthetic biopolymer at sub-ambient temperature (e.g., 4° C.) or ambient temperature (e.g., 20° C.) to physiological temperature (e.g., 37° C.). Preferably, gelation is accompanied by physical cross-linking following ß-sheet formation among molecules of the synthetic biopolymer. That is, the solution undergoes gelation as well as physical cross-linking (e.g., adding desirable structure and strength to the gelation).

Other particular embodiments are directed to a synthetic ELP having a polypeptide sequence comprising functional oligopeptide blocks to achieve desired properties, including any of those described herein that may render the synthetic ELP suitable for use in implantable compositions for tissue repair, such in as bone void filler compositions. Representative synthetic ELPs may have a polypeptide sequence comprising (a) one or more hydrophobic blocks of VPGXG (SEQ ID NO:1), wherein X represents any amino acid other than proline; (b) one or more aggregation-enhancing blocks of IPAVG (SEQ ID NO:2), and (c) one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6). The polypeptide sequence may further comprise: (d) one or more biomineralizing blocks of VTKHLNQISQSY (SEQ ID NO:7) and/or DDDEEKFLRRIGRFG (SEQ ID NO:13).

Further particular embodiments are directed to a composition comprising a synthetic biopolymer or a synthetic ELP as described herein, which composition may be in a solid (e.g., lyophilized) form or an aqueous solution form. In some cases, the composition may be in, or may be manipulated to be in (e.g., following preparation steps, such as hydration, performed prior to use), a paste, putty, or pliable sponge form. Representative compositions may be used in implantation in a human or animal body, such as in the case of a bone void filler composition. Compositions described herein, in general, lack insoluble and/or soluble collagen. For example, one or both forms of collagen may be present in a given composition in an amount, or a combined amount of less than about 10 wt-%, less than about 5 wt-%, or less than about 1 wt-%, based on the total weight of synthetic biopolymer (e.g., polypeptide) present in the composition.

Yet further particular embodiments are directed to a method for preparing a composition comprising a synthetic biopolymer or a synthetic ELP as described herein. The method may comprise: (a) separating the synthetic biopolymer or the synthetic ELP from a cell culture to provide an initial synthesis composition, and optionally performing one or more purification steps; (b) inducing partial (e.g., localized) β-sheet formation among molecules of the synthetic biopolymer or the synthetic ELP, to an extent that does not result in gelation, to provide a post-synthesis treated composition in a solid form or an aqueous solution form; and (c) optionally further processing the post-synthesis treated composition, according to preparation steps, to provide an injectable or implantable composition of the synthetic biopolymer or synthetic ELP. According to such method, the separation from cell culture may be considered a “primary recovery,” performed prior to the purification steps, any formulation steps, and the preparation steps, with step (b) of inducing partial β-sheet formation occurring, in preferred embodiments, after primary recovery and during any of the purification steps and/or formulation steps that provide the post-synthesis treated composition, and/or during any of the preparation steps.

Still further particular embodiments are directed to a method of treating a patient, comprising implanting, in the patient, a composition comprising a synthetic biopolymer or a synthetic ELP as described herein.

Overall, aspects of the invention relate to the discovery of synthetic elastin-like polypeptides and other synthetic biopolymers that have been engineered to undergo physical cross-linking (entanglement of polymer chains and/or formation of interchain β-sheets) in a manner that provides sufficient structure for implantation. The mere use of elastin obtained by known methods (e.g., electrospinning), to produce elastin fibers, would not be satisfactory (e.g., the fibers would collapse in an aqueous environment) in the absence of chemical cross-linking. In contrast, representative synthetic biopolymers described herein do not rely primarily, or at all, on chemical cross-linking, but instead can desirably undergo gelation and physical cross-linking that develops upon increasing temperature. Further advantages of the synthetic elastin-like polypeptides and other synthetic biopolymers as described herein reside in the use of relatively short fibers, in view of the gelling, and optionally mineral particle-retaining, characteristics attained. Implanted collagen fibers, on the other hand, must be of sufficient length to resist “washout” (e.g., when rinsed with cold water by the surgeon) from the site of the tissue defect. Shorter fiber lengths, in turn, translate to additional advantages, in terms of formulating compositions (e.g., after hydration and prior to implantation) having reduced viscosity and/or improved injectability.

These and other aspects, embodiments, and advantages relating to the present invention are apparent from the following Detailed Description.

### BRIEF DESCRIPTION OF THE DRAWINGS

A more complete understanding of exemplary embodiments of the invention and the advantages thereof may be acquired by referring to the following description in consideration of the accompanying figures, which serve to illustrate various features and certain principles involved.

FIGS. 1A, 1B, and 1C are flow diagrams of steps for preparing compositions, according to particular methods as described herein.

FIG. 2 shows the effects of water vapor annealing of samples of synthetic ELPs.

FIG. 3 provides Fourier-transform infrared spectroscopy (FTIR) scans of aqueous solutions of synthetic ELPs, following post-synthesis treatments of freeze-drying alone or in combination with either water vapor annealing or washing with ethanol.

FIG. 4 provides graphs of changes in optical density, resulting from turbidity developed over time for aqueous solutions of synthetic ELPs at varying concentrations, introduced to a photospectrometer preheated at 37° C.

FIG. 5 shows the effects of gelation of samples of synthetic ELPs, following water vapor annealing.

FIG. 6 provides graphs of rheological properties of aqueous solutions of synthetic ELPs, before and after water vapor annealing as a post-synthesis treatment, as such properties are developed over 3 temperature cycles of (i) heating from 4° C. to 37° C. at a heating rate of 1° C. per minute and holding at 37° C. for 30 minutes, followed by (ii) cooling from 37° C. to 4° C. at a cooling rate of 1° C. per minute and holding at 4° C. for 30 minutes.

FIG. 7 provides graphs of rheological properties of aqueous solutions of synthetic ELPs, following various post-synthesis treatments of freeze-drying at different severities, including mild freeze-drying alone or in combination with water vapor annealing, and harsh freeze-drying, as such properties are developed upon heating from 4° C. to 37° C. at a heating rate of 1° C. per minute and holding at 37° C. for 4 hours.

FIG. 8 shows the microstructure of freeze-dried samples of synthetic ELPs.

FIGS. 9A-9C show the effects of biomineralization on samples of synthetic ELPs, with and without biomineralizing blocks in their amino acid sequences.

FIG. 10 provides SDS PAGE analyses of synthetic ELPs, showing that post-synthesis treatments of freeze-drying alone, or in combination with either water vapor annealing or washing with ethanol, have little or no effect on purity or molecular weight of the ELP.

FIG. 11 shows the physical appearance of synthetic ELPs, following post-synthesis treatments of freeze-drying alone, or in combination with either water vapor annealing or washing with ethanol.

FIG. 12 provides dynamic light scattering (DLS) analyses of aqueous solutions of synthetic ELPs, following post-synthesis treatments of freeze-drying alone, or in combination with either water vapor annealing or washing with ethanol, showing the effect of these post-synthesis treatments on particle size, in comparison with denatured ELPs.

FIG. 13 provides graphs of rheological properties of aqueous solutions of synthetic ELPs, following various post-synthesis treatments of freeze-drying alone, or in combination with either water vapor annealing or washing with ethanol, as such properties are developed upon heating from 4° C. to 37° C. at a heating rate of 1° C. per minute and holding at 37° C. for 4 hours.

FIG. 14 provides energy dispersive spectroscopy (EDS) images of films of synthetic ELPs following incubation in different solutions for 1 week at 37° C.

FIG. 15 provides post-dissection oblique, dorsal cut away, and lateral cut away Micro CT images of a tissue defect after implantation in rabbits for 12 weeks.

FIG. 16. Provides graphs of bone volume fraction, mineral implant volume fraction, and bone mineral density after implantation in rabbits for 12 weeks.

### DETAILED DESCRIPTION

Throughout this disclosure, standard, one-letter amino acid codes are used to represent amino acid residues, including their characteristic side chains, in the indicated functional oligopeptide blocks and polypeptides in which they are incorporated. The abbreviation ELP is used for “elastin-like polypeptide” and the abbreviation ELPs is used for “elastin-like polypeptides.” A synthetic ELP is a particular example of a synthetic biopolymer, and therefore, with respect to any description herein that relates to a synthetic biopolymer, this should be understood as relating to a synthetic ELP, in preferred embodiments. Unless otherwise specified, the terms “aqueous solution” and “aqueous composition,” in reference to an aqueous solution or aqueous composition of a synthetic biopolymer (e.g., synthetic ELP), refers to such aqueous solution or aqueous composition having a concentration, in various embodiments, from about 5 mg/ml to about 350 mg/ml, from about 10 mg/ml to about 300 mg/ml, from about 30 mg/ml to about 200 mg/ml, from about 50 mg/ml to about 100 mg/ml, from about 100 mg/ml to about 300 mg/ml, or from about 150 mg/ml to about 250 mg/ml. In more specific embodiments, such concentration of a synthetic biopolymer (e.g., synthetic ELP) in an aqueous solution or aqueous composition as described herein, may be 10 mg/ml, 15 mg/ml, 50 mg/ml, 150 mg/ml, or 250 mg/ml. Exemplary aqueous solutions or aqueous compositions may include, as the aqueous medium, purified water, such as Milli-Q® (MQ) water, or a salt solution, such as phosphate-buffered saline (PBS).

Embodiments of the invention are directed to synthetic biopolymers (e.g., synthetic polypeptides, such as synthetic ELPs) that are engineered, or configured, to undergo gelation when implanted in a human or animal body, a property that renders such synthetic biopolymers, and more specifically compositions formed from such synthetic biopolymers, suitable for tissue repair (e.g., for use in bone void filler compositions). A given synthetic biopolymer may be engineered by virtue of the “design” of its primary structure, or amino acid sequence, particularly in terms of utilizing functional oligopeptide blocks in this sequence. A synthetic biopolymer may alternatively, but preferably in combination, be engineered by its post-synthesis treatment, which may comprise one or more particular steps performed after its recovery from a cell culture (e.g., after primary recovery), and/or after purification. Representative post-synthesis treatments of a synthetic biopolymer may alter, adjust, or tailor its gelation characteristics, such as by influencing, and preferably reducing, the time over which a composition formed from such synthetic biopolymer gels with sufficient structural rigidity as desired for an implant. For example, an aqueous composition of a synthetic biopolymer that has not been subjected to a given post-synthesis treatment and that is used as a control composition, may require a longer period to achieve a given degree of gelation (defined by rheological properties) upon heating according to a given protocol, compared to an aqueous composition of the same synthetic biopolymer, at the same concentration, which has been subjected to the post-synthesis treatment, upon heating according to the same protocol. Therefore, the use of a post-synthesis treatment may advantageously impart properties, and particularly gelation characteristics, that are desired or even necessary for an implant, which characteristics might otherwise be absent without such treatment.

Without being bound by theory, it is believed that physical cross-linking (i.e., without the need for an added cross-linking agent) can occur among molecular chains of synthetic biopolymers described herein according to various mechanisms, which include entanglement of polypeptide strands (e.g., which may be considered a one-dimensional phenomenon) as well as β-sheet formation of polypeptide strands (e.g., which may be considered a two-dimensional phenomenon) through interconnections between strands. In the case of physical cross-linking by β-sheet formation, this may be influenced or engineered, at least in part, in a given synthetic biopolymer by the incorporation of one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6), which are believed to provide these interconnections and/or otherwise influence gelation characteristics. Those skilled in the art having knowledge of the present disclosure will appreciate that the number of physical cross-links that are formed (e.g., by any mechanism) can be “tuned” by adjusting various protocols, including those used for purification and formulation, following primary recovery. With respect to formulation, specific protocols that may be adjusted are those used for drying synthetic polypeptides after purification. Aspects of the invention are related to the discovery that any such adjustments, as part of a given post-synthesis treatment, can significantly influence the gelling behavior of a synthetic biopolymer (e.g., synthetic ELP). In fact, biopolymers having the same amino acid sequence can nonetheless exhibit substantially different gelation characteristics, for example as evidenced by gelling behavior determined experimentally at physiological temperature (e.g., over a range of not gelling whatsoever to forming gels with storage moduli of significantly greater than 10 kPa), depending on the extent of β-sheet formation post-synthesis, such as during purification and formulation (e.g., drying, and typically freeze-drying). According to particular embodiments, a baseline, or possibly lowest, content of β-sheets may be achieved (induced) utilizing a relatively “harsh” freeze-drying step as described herein. The obtained, freeze-dried synthetic biopolymer may then be subjected, for example, to water vapor annealing, or may then otherwise be subjected to washing with an organic liquid (e.g., ethanol), either of which additional post-synthesis treatment steps can further induce the formation of β-sheets from β-sheet formation-inducing blocks as described above (e.g., GAGAGS (SEQ ID NO:3)) in the polypeptide sequence of the synthetic biopolymer. The recognition that post-synthesis treatments can influence physical cross-linking, at least partly resulting from β-sheet formation, provides novel methods for monitoring and/or tuning mechanical properties of synthetic biopolymers to achieve desired outcomes for practical applications.

Representative post-synthesis treatments may therefore affect the secondary structure of the synthetic biopolymer and may, more particularly, cause at least some j-sheet formation among molecules of the synthetic biopolymer, for example due to the presence in these molecules of β-sheet formation-inducing block(s) (“silk” blocks) as noted above. This, in turn, can initiate, or template, physical cross-linking among these molecules (e.g., following implantation), such as by proceeding through a process of assembly of formed β-sheets, optionally in combination with entanglement of individual polypeptide strands, thereby forming physically cross-linked networks of supramolecular fibers. Advantageously, a post-synthesis treatment can be used to engineer a synthetic biopolymer, by induction of at least a portion of the β-sheet formation that ultimately accompanies gelation and that can beneficially influence the structure and strength of that gelation. Such induction can therefore effectively facilitate obtaining these desired, subsequent gelation characteristics of a synthetic biopolymer or composition comprising this synthetic biopolymer, upon exposure to elevated temperature, for example by heating of a solution of the synthetic biopolymer at sub-ambient temperature to physiological temperature. Although the post-synthesis treatment itself generally does not result in gelation, it is possible that induction of β-sheet formation by such treatment may result in physical cross-linking and/or other changes that precede and/or initiate physical cross-linking, including some filament assembly (entanglement) as described herein. A post-synthesis treatment may result in differences in properties of compositions comprising a given synthetic biopolymer, such as an increase in viscosity of an aqueous solution following such treatment, relative to that prior to such treatment. In some embodiments, β-sheet formation due to a post-synthesis treatment can be detected or confirmed using Fourier-transform infrared spectroscopy (FTIR) to scan compositions, and preferably aqueous compositions, with and without (e.g., following and prior to) such treatment. Physical cross-linking, at least partly resulting from β-sheet formation, may be induced by a given post-synthesis treatment, which in turn may comprise one or more specific post-synthesis treatment steps as described herein. This physical cross-linking, in addition to, or alternatively to, increased viscosity of an aqueous solution as noted above, may result in increased particle size in such solution. This may be detected based on a measured average particle size of a composition comprising a synthetic biopolymer (e.g., synthetic ELP) as described herein. According to other embodiments, therefore, β-sheet formation due to a post-synthesis treatment can be detected or confirmed using a suitable analytical method for measuring average particle size, such as dynamic light scattering (DLS), to analyze compositions, and preferably aqueous compositions, with and without (e.g., following and prior to) a given post-synthesis treatment. To the extent a post-synthesis treatment, comprising one or more post-synthesis treatment steps as described herein, influences interactions among molecules, its effects may be more accurately characterized as applying to a composition comprising the synthetic biopolymer, as opposed to a molecule of the synthetic biopolymer itself. In this respect, such characterization may therefore differ from that of the amino acid sequence, which is specific to the molecule.

According to particular embodiments, a synthetic biopolymer may be engineered (e.g., according to adaptations of its primary and/or secondary structure) to undergo gelation, following heating of a solution of the synthetic biopolymer at sub-ambient temperature (e.g., 4° C.) or ambient temperature (20° C.) to physiological temperature (e.g., 37° C.). Such gelation characteristics, or other gelation characteristics, may serve as a proxy for evaluating the performance of such synthetic biopolymer for its practical use in a composition designed for implantation. In this regard, to the extent that gelation characteristics may, in general, be influenced by physical cross-linking following β-sheet formation among molecules of the synthetic biopolymer, any of the gelation characteristics as described with respect to specific protocols defined herein (e.g., rheological properties obtained upon, or after, heating of a composition or subjecting a composition to one or more temperature cycles) may be used as a basis for characterization of a given synthetic biopolymer, regardless of the particular mechanism whereby such gelation characteristics are achieved.

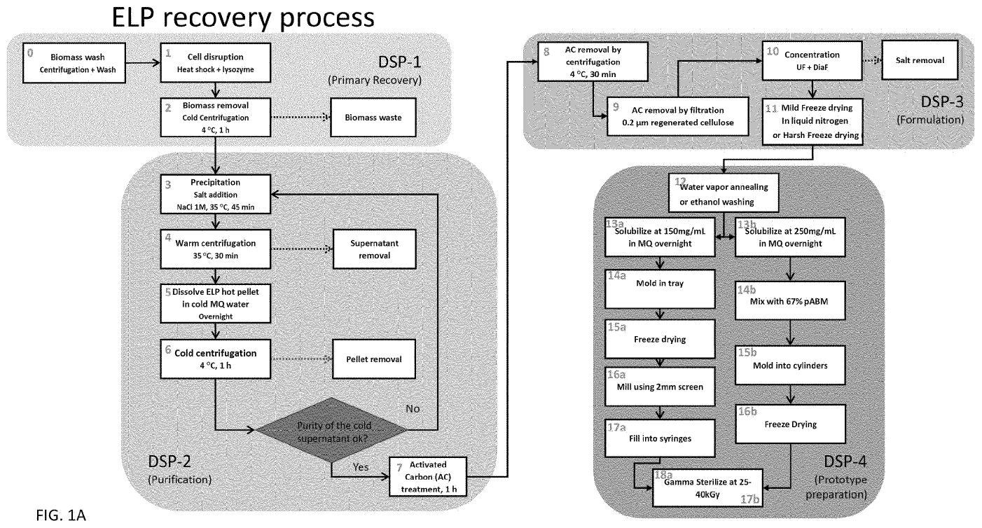

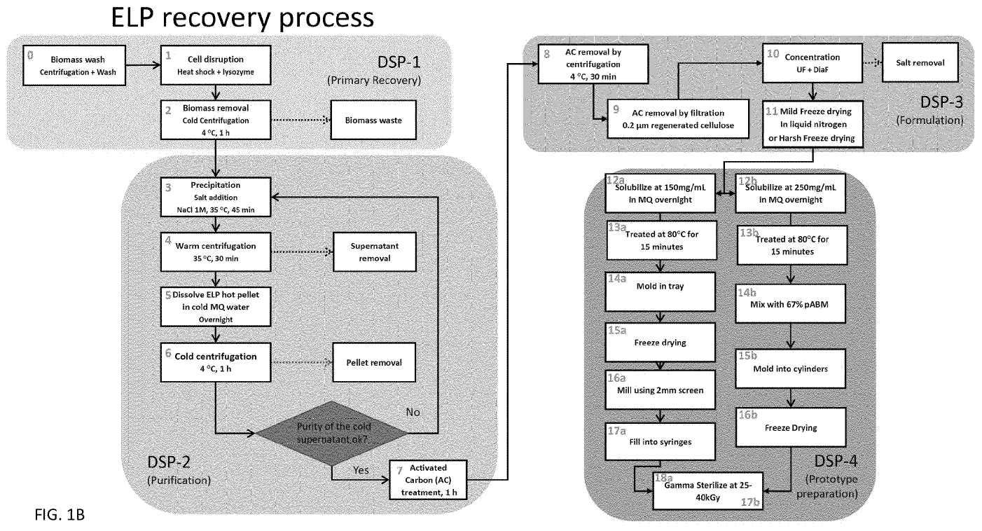

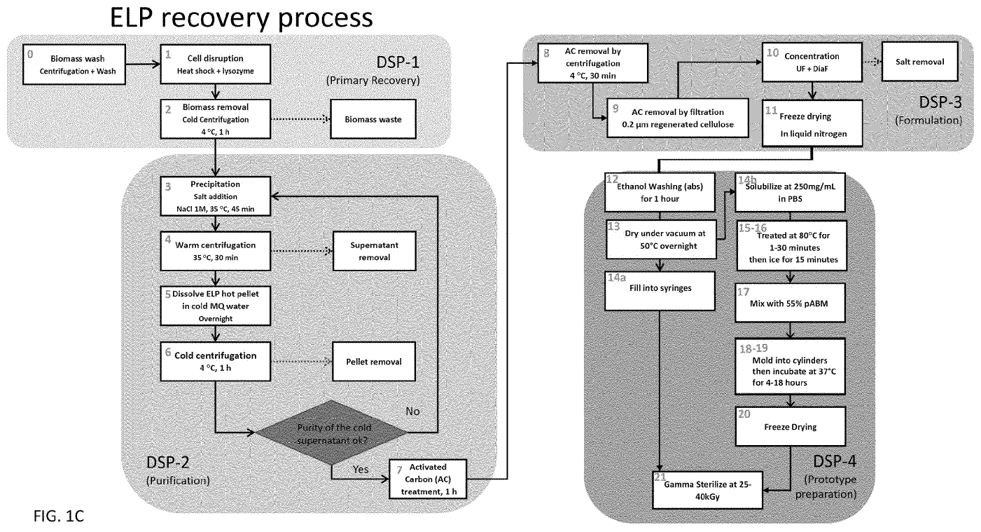

Particular examples of post-synthesis treatments, which can be used to engineer a given synthetic biopolymer by imparting characteristics as described herein, can include steps of (i) freeze-drying, (ii) water vapor annealing, (iii) washing with an organic liquid, and/or (iv) thermal exposure. Combinations of such treatment steps are also possible, such as in the case of freeze-drying, followed by water vapor annealing. A post-synthesis treatment may include any one or more of such post-synthesis treatment steps to influence gelation characteristics, with the term “post-synthesis” referring to steps occurring following the preparation of an initial synthesis composition of the synthetic biopolymer, such as following its separation and recovery from a cell culture. Typically, this separation and recovery can involve steps such as centrifugation, cell rupturing, and sonication. In one embodiment, with reference to FIGS. 1A 1B, and 1C, post-synthesis treatment refers to treatment steps occurring following the “Primary recovery” block, and typically within the “Purification,” “Formulation,” and “Prototype preparation” blocks. Often, post-synthesis treatment may be manipulated solely within formulation of the synthetic biopolymer and subsequent processing steps, and not within purification. For example, the step of (i) may involve the use of, and/or manipulation of, freeze-drying that occurs as part of formulation. The steps (ii) and/or (iii) may involve the use of, and/or manipulation of, water vapor annealing and/or washing with an organic liquid that may occur subsequent to a drying step (e.g., freeze-drying) that occurs as part of formulation. In the same manner, the step (iv) thermal exposure may be used and/or manipulated subsequent to a drying step, as described herein. Alternatively, or in combination, the step (iv) can include the manner in which the synthetic biopolymer is thermally exposed prior to a drying (e.g., freeze-drying) step. For example, with reference to FIGS. 1A, 1B, and 1C, thermal exposure may include the manipulation of the “Warm centrifugation” step and more particularly the specific temperature, or the specific time-temperature profile, to which the synthetic biopolymer is exposed during this step. In this manner, and more generally, the step (iv), which may be used and/or manipulated to influence gelation characteristics as described herein, may comprise one or both of (1) a pre-drying thermal exposure and (2) a post-drying thermal exposure, with the recognition according to the present disclosure that thermal exposure occurring over the entire processing of a given synthetic biopolymer (prior to its end use) can influence gelation characteristics. According to particular embodiments, a pre-drying thermal exposure may occur before freeze-drying (e.g., in liquid nitrogen) and/or a post-drying thermal exposure may occur after freeze-drying. In other particular embodiments, a pre-drying thermal exposure or a post-drying thermal exposure, for example occurring before or after freeze-drying, may itself involve drying of a composition of a synthetic biopolymer, such as in the case of drying at elevated temperature. In this regard, FIG. 1C more specifically illustrates a step 13 of drying under vacuum at 50° C., following a step 12 of washing with an organic liquid. Insofar as step 13 occurs after freeze-drying, it may be considered a post-drying thermal exposure. It can also be appreciated that steps 12 and 13 of FIG. 1C represent post-synthesis treatments steps that are namely preparation steps (occurring in the illustrated “prototype preparation” block) for further processing the post-synthesis treated composition, with these steps having the effect of inducing physical cross-linking such as by 3-sheet formation as described herein.

Important aspects of the invention relate to finding that a post-synthesis treatment, and specific post-synthesis treatment steps such as (i), (ii), (iii), and/or (iv) as described herein, can affect measurable properties that are indicative of an extent of physical cross-linking, such as occurring at least partly by β-sheet formation. These properties include, for example, viscosity, infrared absorption spectrum (e.g., FTIR spectrum), average particle size and/or particle size distribution, and gelation characteristics, any of which properties may be determined as described herein (e.g., by a suitable analysis of an aqueous solution of a given synthetic biopolymer). Accordingly, in representative methods, a post-synthesis treatment may be adjusted or modified, based on a property of a composition comprising a synthetic biopolymer as described herein, wherein such property is indicative of an extent of physical cross-linking, such as occurring at least partly by β-sheet formation. For example, according to some embodiments, in the case of an indication of low physical cross-linking or β-sheet formation (such as determined by the property of gelation characteristics that are insufficient), water-vapor annealing, washing with an organic liquid, and/or a post-drying thermal exposure may be used or manipulated (e.g., to increase severity). According to other embodiments, in the case of an indication of low physical cross-linking or β-sheet formation (for example as determined by the property of gelation characteristics that are insufficient), freeze-drying and/or a pre-drying thermal exposure may be manipulated (e.g., to increase severity). According to yet other embodiments, in the case of an indication of high physical cross-linking or β-sheet formation (for example as determined by the property of gelation characteristics that are excessive), water-vapor annealing, washing with an organic liquid, and/or a post-drying thermal exposure may be eliminated or manipulated (e.g., to decrease severity). According to still other embodiments, in the case of an indication of high physical cross-linking or β-sheet formation (for example as determined by the property of gelation characteristics that are excessive), freeze-drying and/or a pre-drying thermal exposure may be manipulated (e.g., to decrease severity). With respect to such embodiments, in the case of a post-synthesis treatment step being “used,” this refers to the implementation of such step to achieve an extent of physical cross-linking or β-sheet formation, or otherwise achieve gelation characteristics, where such post-synthesis treatment step is/was not used in a baseline, comparative, or previous post-synthesis treatment. In the case of a post-synthesis treatment step being “eliminated,” this refers to the removal of such step to achieve an extent of physical cross-linking or β-sheet formation, or otherwise achieve gelation characteristics, where such post-synthesis treatment step is/was used in a baseline, comparative, or previous post-synthesis treatment. In the case of a post-synthesis treatment step being “manipulated,” this refers to a change in severity, relative to that of the same post-synthesis step that is/was used in a baseline, comparative, or previous post-synthesis treatment. Those skilled in the art having knowledge of the present disclosure will appreciate how post-synthesis treatment steps can be manipulated to increase or decrease severity (e.g., increase a time and/or temperature of thermal exposure as described herein) and thereby regulate, i.e., increase or decrease, the extent of physical cross-linking or β-sheet formation. Embodiments of the invention are therefore directed to methods for engineering synthetic biopolymers as described herein, to achieve desired gelation characteristics, for example based on rheology measurements as described herein, with such methods comprising adjusting or modifying protocols used for preparing compositions comprising these synthetic biopolymers. The adjusting or modifying can comprise the use, manipulation, or elimination of particular post-synthesis treatment steps as described herein.

It can therefore be appreciated that a post-synthesis treatment, such as comprising one or more post-synthesis treatment steps that may include (i) freeze-drying; (ii) water vapor annealing; (iii) washing with an organic liquid; and/or (iv) thermal exposure, which may more specifically comprise one or both of (1) a pre-drying thermal exposure and/or (2) a post-drying thermal exposure; may be adjusted or modified, such as in the case of these one or more post-synthesis treatment steps being used and/or manipulated as described above, based on one or more properties indicative of an extent of physical cross-linking or β-sheet formation. Such properties include gelation characteristics, for example those a given aqueous solution of a synthetic biopolymer (e.g., synthetic ELP), which may be used as a basis for a post-synthesis treatment being adjusted or modified. Examples of determinations of gelation characteristics as being either insufficient or excessive are described herein.

Other properties, in response to which (A1) water-vapor annealing, washing with an organic liquid, and/or a post-drying thermal exposure may be used or manipulated (e.g., to increase severity), and/or (A2) freeze-drying and/or a pre-drying thermal exposure may be manipulated (e.g., to increase severity), include viscosity, infrared absorption spectrum (e.g., FTIR spectrum), average particle size and/or particle size distribution. These properties may likewise be the basis for a response according to which (B1) water-vapor annealing, washing with an organic liquid, and/or a post-drying thermal exposure may be eliminated or manipulated (e.g., to decrease severity), and/or (B2) freeze-drying and/or a pre-drying thermal exposure may be manipulated (e.g., to decrease severity). For example, a viscosity of a standard solution, such as an aqueous solution of a synthetic biopolymer (e.g., synthetic ELP) at a specific concentration and temperature, being below a threshold minimum viscosity, may be the basis for a response A1 and/or A2, whereas such viscosity being above a threshold maximum viscosity may be the basis for a response B1 and/or B2. According to other embodiments, an infrared absorption spectrum (e.g., FTIR spectrum) of a standard solution having an absorbance at a given wavenumber (e.g., at about 1622 cm⁻¹) being below a threshold minimum absorbance may be the basis for a response A1 and/or A2, whereas such absorbance being above a threshold maximum absorbance may be the basis for a response B1 and/or B2. According to yet other embodiments, with respect to an average particle size or a percentage of particles above a given particle size (e.g., in the case of a bimodal or multi-modal particle size distribution), such as measured in an aqueous solution of a synthetic biopolymer (e.g., synthetic ELP), in the case of such average particle size being below a threshold minimum particle size or such percentage being below a minimum threshold percentage, this may be the basis for a response A1 and/or A2, whereas in the case of such average particle size being above a threshold maximum particle size or such percentage being above a maximum threshold percentage, this may be the basis for a response B1 and/or B2. For example, according to particular embodiments, a threshold minimum particle size may be any discreet value within the range of 10 nanometers (nm) to 100 nm; a threshold maximum particle size may be any discreet value within the range of 100 nm to 500 nm; a minimum threshold percentage may be any discreet percentage within the range of 10% to 90%, representing the percentage of particles having a particle size above any discreet value within the range of 10 nm to 100 nm; and a maximum threshold percentage may be any discreet percentage within the range of 10% to 90%, representing the percentage of particles having a particle size above any discreet value within the range of 100 nm to 500 nm. Particle size and particle size distribution may be measured, for example, using DLS. Those skilled in the art having knowledge of the present disclosure will appreciate other specific adjustments/modifications to post-synthesis treatments that may be made in response to determinations of one or more properties indicative of physical cross-linking or β-sheet formation.

In the case of freeze-drying, this post-synthesis treatment step may comprise freezing an aqueous solution of the synthetic biopolymer and drying the frozen solution under vacuum pressure to sublimate frozen water. The severity of freeze-drying can be controlled by adjusting, for example, the surface area of frozen solution that is exposed to the vacuum conditions, the surface area-to-volume ratios of frozen volumes of the aqueous solution, the concentration of the solution, and the drying temperature. According to some embodiments, freeze-drying may, alone, be sufficient for induction of β-sheet formation to a desired extent, such that water vapor annealing or other post-synthesis treatment step, according to a given post-synthesis treatment, may not be required. According to other embodiments, freeze-drying may be used in combination with water vapor annealing, washing with an organic liquid, and/or other post-synthesis treatment step, according to a given post-synthesis treatment, to achieve induction of β-sheet formation to a desired extent, for example as determined based on any one or more properties as described herein.

A relatively more severe or “harsh” freeze-drying may comprise subjecting discreet, relatively large, frozen volumes (e.g., 35 ml frozen portions in plastic tubes) to uncontrolled temperature (e.g., ambient or room temperature) drying, whereas a relatively less severe or “mild” freeze-drying may comprise subjecting discreet, relatively small, frozen volumes (e.g., 50 μl droplets) to drying over a period, at least some portion of which (e.g., the majority of the drying period) is conducted at above-ambient temperature (e.g., 30° C.). In the case of the mild freeze-drying, the surface area-to-volume ratio of the frozen volumes is significantly higher than that as described with respect to the harsh freeze-drying. This higher surface area-to-volume ratio, combined with higher drying temperatures and/or less vacuum, may result in an overall more homogeneous process. In some cases, a mild freeze-drying can avoid the formation of a dense crust at the outer periphery of the resulting, freeze-dried volumes, which is a source of non-homogeneity that can be undesirable. According to a specific embodiment, in the case of harsh freeze-drying, 50 ml plastic tubes, each containing frozen 35 ml volumes of aqueous solution of synthetic biopolymer, are placed in a freeze-dryer maintained at 0.05 millibar (mbar) absolute pressure with a condenser temperature of −80° C. and allowed to dry for 48 hours at room temperature. According to another specific embodiment, this harsh freeze-drying may be modified to mild freeze-drying, according to which frozen droplets of the aqueous solution, for example obtained by dropwise addition of the aqueous solution into liquid nitrogen and subsequent filtration, are placed in a freeze-dryer (e.g., in a tray) maintained at 0.5 millibar (mbar) absolute pressure with a condenser temperature of −90° C., and allowed to dry according to a pre-programmed procedure, such as at a shelf (surrounding) temperature of 15° C. for 10 hours and 30° C. for 24 hours. Those skilled in the art having knowledge of the present disclosure will appreciate the variables that impact freeze-drying severity and consequently the conditions (e.g., surface area-to-volume ratio, pressure, and drying temperature) that can be altered to achieve a desired degree of homogeneity and other desired characteristics of a freeze-dried composition of a given synthetic biopolymer, considering the overall economics associated with the time and conditions required for freeze-drying under varying levels of severity.

In the case of water vapor annealing, this post-synthesis treatment may comprise exposing the synthetic biopolymer (e.g., in a solid form, such as a lyophilized form after being subjected to freeze-drying as described herein) to water vapor under vacuum conditions. For example, synthetic biopolymer in the form of a “fluffy” solid or other solid form of this material may be positioned above a water reservoir, such as placed in a tray on a support structure (e.g., disk) having multiple holes and mounted above this reservoir. A vacuum desiccator, charged with a volume of water below such support structure, is an exemplary apparatus that may be used for this purpose. Under vacuum pressure, the water evaporates and increases the surrounding humidity, causing water adsorption into the synthetic biopolymer, such that its weight may increase, for example, by at least about 50%, at about least 75%, or at least about 100%, relative to an initial weight prior to the water vapor annealing. In general, water vapor annealing also includes subsequent drying, for example in ambient air, thereby reducing moisture content. According to particular embodiments, water vapor annealing may be used as a post-synthesis treatment alone, or otherwise in combination with freeze-drying (e.g., by performing water vapor annealing before or after freeze-drying, and preferably after), for induction of β-sheet formation to a desired extent (e.g., as determined by FTIR and/or other properties as described herein, which are indicative of an extent of physical cross-linking or β-sheet formation). In the case of post-synthesis treatment comprising freeze-drying followed by water vapor annealing, the latter step may serve to further induce β-sheet formation, beyond an extent induced by the former step. Whether employed alone or in combination with one or more other post-synthesis treatment steps, and without being bound by theory, it is believed that water vapor annealing induces (or further induces) β-sheet formation as a result of the water adsorption that occurs, which imparts “plasticity” to molecules of the synthetic biopolymer, allowing them to become mobile and align β-sheet forming regions.

A further example of a post-synthesis treatment, which may be used as an alternative to one or both of freeze-drying or water vapor annealing, or which otherwise may be used in combination with one or both of these, is thermal exposure, which can likewise induce β-sheet formation when used alone, or otherwise further induce β-sheet formation in combination with other steps as described herein. In one embodiment, for example, thermal exposure can substitute for water vapor annealing, as a post-synthesis treatment step performed following freeze-drying. Like water vapor annealing, thermal exposure is also believed to increase mobility among molecules of the synthetic biopolymer, thereby allowing them to align β-sheet forming regions. Thermal exposure may comprise heating an aqueous solution of a synthetic biopolymer to an elevated temperature, generally above physiological temperature, that does not cause significant degradation or denaturing of the synthetic biopolymer. In representative embodiments, thermal exposure may comprise heating an aqueous solution of a synthetic biopolymer at a concentration from about 10 mg/ml to about 300 mg/ml, and preferably from about 100 mg/ml to about 300 mg/ml (e.g., 150 mg/ml or 250 mg/ml) to a temperature in a range from about 35° C. to about 100° C., preferably from about 50° C. to about 90° C., and more preferably from about 70° C. to about 85° C. (e.g., 80° C.). The temperature within this range (or temperature at this value) may be maintained for a thermal exposure time period sufficient for induction (or further induction) of β-sheet formation to a desired extent. In representative embodiments, this thermal exposure time period is from about 1 minute to about 12 hours, from about 1 minute to about 8 hours, from about 1 minute to about 60 minutes, from about 5 minutes to about 45 minutes, or from about 10 minutes to about 30 minutes. In general, thermal exposure may benefit from subsequent drying, for example in ambient air, thereby reducing moisture content and volume of the resulting composition (e.g., post-synthesis treated composition in an aqueous solution form).

According to particular embodiments, thermal exposure comprising heating of an aqueous solution of a synthetic biopolymer, as described above, may be a post-drying thermal exposure, i.e., performed following drying (e.g., freeze-drying) that occurs during formulation. Temperatures and times for post-drying thermal exposure include those in the ranges as described above, and this post-synthesis treatment step more broadly comprises the use of a post-drying temperature of at least about 35° C., such as at least about 50° C., and a post-drying exposure time of at least about 1 minute. According to other embodiments, thermal exposure may be more particularly a pre-drying thermal exposure, i.e., performed prior to drying (e.g., freeze-drying), such as in the case of being performed during purification (e.g., during warm centrifugation) and/or during formulation. This post-synthesis treatment step broadly comprises the use of a pre-drying temperature of at least about 35° C. (e.g., from about 35° C. to about 50° C.) and a pre-drying exposure time of at least 1 about minute (e.g., within any of the ranges described above with respect to thermal exposure time periods). With respect to manipulating a pre-drying thermal exposure and/or post-drying thermal exposure as described above, such as to increase or decrease its severity based on any one or more properties indicative of an extent of physical cross-linking or β-sheet formation, directionally increasing severity can be performed in either case by increasing the time and/or temperature of exposure (e.g., within any of the thermal exposure time ranges and/or thermal exposure temperature ranges described above), whereas directionally decreasing severity can be performed in either case by decreasing the time and/or temperature of exposure (e.g., within any of the thermal exposure time ranges and/or thermal exposure temperature ranges described above). For example, a pre-drying thermal exposure step can be made more severe by increasing the temperature of warm centrifugation, as part of synthetic biopolymer purification, and/or increasing the time over which this step of purification is performed. In general, the manipulation of a pre-drying thermal exposure step, and/or the use or manipulation of a post-drying thermal exposure step, can be performed as part of a post-synthesis treatment used to affect the extent of physical cross-linking or β-sheet formation, which in turn can be determined based on various properties (e.g., gelation characteristics, viscosity, infrared absorption spectrum, particle size and/or particle size distribution) as described herein.

A further example of a post-synthesis treatment, which may be used as an alternative to freeze-drying, water vapor annealing, and/or thermal exposure, or which otherwise may be used in combination with one or more of these, is washing with an organic liquid, which can likewise induce β-sheet formation when used alone, or otherwise further induce β-sheet formation in combination with other steps as described herein. In various embodiments, for example, washing with an organic liquid can substitute for water vapor annealing, as a post-synthesis treatment step performed following freeze-drying. Otherwise, washing with an organic liquid can be used in combination with water vapor annealing. Like water vapor annealing, washing with an organic liquid is also believed to result in dehydration of the synthetic biopolymer, as a possible mechanism for inducing β-sheet formation. Therefore, for example, a representative post-synthesis treatment may comprise drying (e.g., freeze-drying), optionally further in combination with water vapor annealing or washing with an organic liquid, or otherwise optionally further in combination with both of these post-synthesis treatment steps (e.g., water vapor annealing, followed by washing with an organic liquid).

Washing with an organic liquid may comprise contacting, for example in a batchwise or continuous manner, a dried (e.g., freeze-dried) form of the synthetic biopolymer with any suitable organic liquid that does not adversely react with the synthetic biopolymer. A representative organic liquid may be selected from the group consisting of an alcohol (e.g., methanol, ethanol, propanol, and butanol), a hydrocarbon (e.g., a C₄-C₈ alkane hydrocarbon), an ether (e.g., a dialkyl ether having C₁-C₄ alkyl groups), a carboxylic acid (e.g., having from 2 to 6 carbon atoms), an ester (e.g., having from 2-6 carbon atoms), and a ketone (e.g., dialkyl ketone having C₁-C₄ alkyl groups). A preferred organic liquid is ethanol. Representative temperatures and contacting times (e.g., residence times in the case of continuous contacting) include, respectively, approximately ambient temperature, such as from about 15° C. to about 35° C. and a range from about 1 minute to about 24 hours, such from about 1 hour to about 12 hours. Preferably, washing with an organic liquid also includes subsequent drying, for example in the case of air-drying for a time from about 1 hour to about 24 hours at approximately ambient or elevated temperature, such as from about 15° C. to about 80° C., or from about 15° C. to about 50° C., optionally under vacuum. Alternatively, as noted above with respect to FIG. 1C, such subsequent drying may otherwise be considered a separate post-synthesis treatment step of thermal exposure, which can be manipulated (e.g., by increasing or decreasing severity) to induce physical cross-linking, such as by β-sheet formation as described herein, to a desired extent. In either case, the ability of a given washing step to cause dehydration may be monitored and/or verified by measuring the moisture content of the synthetic biopolymer, for example before and after washing with the organic liquid, or otherwise monitoring vapors being driven off during the subsequent drying. A step of washing with an organic liquid may have the added benefit of removing impurities (e.g., by extracting organic compounds), such as anti-foaming agents, which may have been used during prior purification and/or formulation of the synthetic biopolymer.

In general, post-synthesis treatments, which may include one or more steps as described herein, can be used to engineer desired gelation characteristics as described herein with respect to specific protocols. Regardless of which particular treatment is employed, the induction of β-sheet formation to a desired extent may be confirmed by analytical methods for determining properties such as those described herein, including gelation characteristics and/or other properties indicative of an extent of physical cross-linking or β-sheet formation, including viscosity, infrared absorption spectrum (e.g., FTIR spectrum), or average particle size and/or particle size distribution. Such methods may be performed on a given synthetic biopolymer, or more precisely a sample thereof, obtained following its formulation, such as subsequent to drying (e.g., freeze-drying) and optionally other post-synthesis treatment steps as described herein. Those skilled in the art and having knowledge of the present disclosure can determine, for a given synthetic biopolymer, post-synthesis treatment steps and associated conditions (severity) as needed to attain an extent of induction of β-sheet formation, to influence gelation characteristics in a desired manner.

Additional embodiments of the invention are directed to synthetic ELPs having a polypeptide sequence comprising defined, functional oligopeptide blocks that are effective in synthetic biopolymers generally, for providing advantageous characteristics as described herein. Representative synthetic ELPs have a polypeptide sequence comprising: (a) one or more hydrophobic blocks of VPGXG (SEQ ID NO: 1), wherein X represents any amino acid other than proline; (b) one or more aggregation-enhancing blocks of IPAVG (SEQ ID NO:2); and (c) one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6). According to more particular embodiments, X in the one or more hydrophobic blocks of VPGXG (SEQ ID NO:1), such as in a portion of these hydrophobic blocks, or in all of the hydrophobic blocks, (i) may represent V, I, or E, or (ii) may represent V or I. For example, at least about 50%, at least about 60%, at least about 70%, or at least about 80% of the one or more hydrophobic blocks of VPGXG (SEQ ID NO:1) may be VPGVG (SEQ ID NO:8) and, optionally, the remainder of these blocks may be VPGIG (SEQ ID NO:9) or VPGEG (SEQ ID NO:10). The hydrophobic blocks VPGVG (SEQ ID NO:8) and VPGIG (SEQ ID NO:9), in particular, have been found to significantly affect hydrophobicity of the synthetic ELP and consequently the gelation temperature.

In some embodiments, the one or more hydrophobic blocks of VPGXG (SEQ ID NO:1) (e.g., one or more hydrophobic blocks of either VPGVG (SEQ ID NO:8) or VPGIG (SEQ ID NO:9)), may be from about 1 to about 150, from about 5 to about 120, from about 5 to about 100, or from about 25 to about 75, of these blocks. With respect to any of these ranges of hydrophobic blocks of VPGXG (SEQ ID NO: 1), alternative values for the maximum stated value are 75, 60, 50, 40, 35, and 30. Therefore, for example, the above-recited range of about 1 to about 150, in alternative embodiments, may be from about 1 to about 75, from about 1 to about 60, from about 1 to about 50, from about 1 to about 40, from about 1 to about 35, or from about 1 to about 30. In other exemplary embodiments, representative synthetic ELPs may comprise (i) from about 1 to about 100, from about 1 to about 75, or from about 5 to about 60, hydrophobic blocks of VPGVG (SEQ ID NO:8) and/or (ii) from about 1 to about 80, from about 1 to about 50, or from about 2 to about 25, hydrophobic blocks of VPGIG (SEQ ID NO:9).

Alternatively, or in combination with such numbers of hydrophobic blocks and/or such percentages of particular hydrophobic blocks, the one or more aggregation-enhancing blocks of IPAVG (SEQ ID NO:2) in representative synthetic ELPs may be from about 1 to about 50, from about 1 to about 30, from about 5 to about 50, or from about 5 to about 20, of these blocks.

Alternatively, or in combination with such numbers of hydrophobic blocks, such percentages of particular hydrophobic blocks, and/or such numbers of aggregation-enhancing blocks, the one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6) in representative synthetic ELPs may be from about 1 to about 50, from about 1 to about 30, or from about 5 to about 20, of these blocks. Preferably, at least about 75%, at least about 90%, or possibly all, of the one or more β-sheet formation-inducing blocks are GAGAGS (SEQ ID NO:3). The number and/or percentage of particular β-sheet formation-inducing blocks can be used to influence the extent of β-sheet formation among molecules of the synthetic ELP, when subjected to a given condition, such as a post-synthesis treatment as described herein. In some embodiments, β-sheet formation-inducing blocks (e.g., GAGAGS; SEQ ID NO:3) may represent from about 1 wt-% to about 50 wt-%, from about 3 wt-% to about 40 wt-%, from about 5 wt-% to about 35 wt-%, or from about 10 wt-% to about 20 wt-%, of the total weight of the synthetic ELP (i.e., the combined molecular weight of these functional oligopeptide blocks may represent these percentages of the total molecular weight of the synthetic ELP).

Alternatively, or in combination with such numbers of hydrophobic blocks, such percentages of particular hydrophobic blocks, such numbers of aggregation-enhancing blocks, such numbers of β-sheet formation-inducing blocks, and/or such percentages of particular β-sheet formation-inducing blocks, one or both ends of representative synthetic ELPs (i.e., a first end and/or a second end, meaning one or both termini of the molecule) may be formed exclusively by (i) at least a portion of (b) the one or more aggregation-enhancing blocks of IPAVG (SEQ ID NO:2), and/or (ii) at least a portion of (c) the one or more s-sheet formation-inducing blocks of GAGAGS (SEQ ID NO:3), GAGAGY (SEQ ID NO:4), GAGYGA (SEQ ID NO:5), or GAGAGA (SEQ ID NO:6). For example, the one or both ends may be formed exclusively by (i) at least a portion of (b) the one or more aggregation-enhancing blocks of IPAVG (SEQ ID NO:2), and/or (ii) at least a portion of (c) the one or more β-sheet formation-inducing blocks of GAGAGS (SEQ ID NO:3) in particular. It can be appreciated that an end formed exclusively by a combination of (i) and (ii) represents either a specific embodiment of this end being formed exclusively by (i), or a specific embodiment of this end being formed exclusively by (ii). For example, an end formed by (IPAVG, SEQ ID NO:2)ₓ(GAGAGS, SEQ ID NO:3)_{y}(VPGVG, SEQ ID NO:8)_{z}, in which x, y, and z are positive integers, is an example of an end formed exclusively by a combination of (i) and (ii), and this example is a specific embodiment an end formed exclusively by (i). In some cases, one end of a representative synthetic ELP may be formed exclusively by (i) at least a portion of the one or more aggregation-enhancing blocks of IPAVG (SEQ ID NO:2), and the opposite end may be formed exclusively by (ii) at least a portion of the one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6), and preferably one or more of GAGAGS (SEQ ID NO:3).

Optionally, with respect to any synthetic ELP comprising functional oligopeptide blocks as defined above, and in particular embodiments with respect to synthetic ELPs having, as defined above, such numbers of hydrophobic blocks, such percentages of particular hydrophobic blocks, such numbers of aggregation-enhancing blocks, such numbers of β-sheet formation-inducing blocks, such percentages of particular β-sheet formation-inducing blocks, and/or such characteristics of one or both ends, such synthetic ELPs may optionally further comprise (d) one or more biomineralizing blocks of VTKHLNQISQSY (SEQ ID NO:7) and/or DDDEEKFLRRIGRFG (SEQ ID NO:13). The one or more biomineralizing blocks in representative synthetic ELPs may be from about 1 to about 25, from about 1 to about 20, from about 1 to about 15, or from about 1 to about 10, of these blocks. Advantageously, it has been found that such biomineralizing blocks can improve the properties of synthetic ELPs, in terms of their ability to form compositions that, in the physiological environment, sequester and/or retain constituent ions of bone mineral, namely phosphate and calcium ions, for applications as described herein (e.g., implantation to repair tissue damage, including bone defects). Compositions comprising synthetic ELPs having biomineralizing block(s) may advantageously further comprise mineral particles as described herein (e.g., hydroxyapatite and/or calcium phosphate, such as ß-tricalcium phosphate). The presence of these mineral particles, in combination with the functionality of biomineralizing block(s) to sequester phosphate ions from the bloodstream, which in turn attract and sequester calcium ions, is believed to facilitate bone growth through the mineralization process, according to which bioavailable calcium and phosphate lead to the precipitation of hydroxyapatite bone mineral.

With respect to any synthetic ELP comprising functional oligopeptide blocks as defined herein, and in particular embodiments with respect to synthetic ELPs having, as defined herein, such numbers of hydrophobic blocks, such percentages of particular hydrophobic blocks, such numbers of aggregation-enhancing blocks, such numbers of β-sheet formation-inducing blocks, such percentages of particular β-sheet formation-inducing blocks, and/or such numbers of biomineralizing blocks, these synthetic ELPs may have a molecular weight from about 10 kilo Daltons (kDa) to about 100 kDa, from about 15 kDa to about 60 kDa, from about 20 kDa to about 50 kDa, or from about 25 kDa to about 40 kDa and/or these synthetic ELPs may have an isoelectric pH, or isoelectric point pI value, from about 4 to about 11 or from about 5 to about 10.

With respect to any synthetic ELP comprising functional oligopeptide blocks as defined herein, and in particular embodiments with respect to synthetic ELPs having, as defined herein, such numbers of hydrophobic blocks, such percentages of particular hydrophobic blocks, such numbers of aggregation-enhancing blocks, such numbers of β-sheet formation-inducing blocks, such percentages of particular β-sheet formation-inducing blocks, and/or such numbers of biomineralizing blocks, these synthetic ELPs may consist of, or consist essentially of, functional oligopeptide blocks (a), (b), (c), and optionally (d), as defined herein. In some embodiments, functional oligopeptide blocks (a), (b), (c), and optionally (d), as defined herein, may represent at least about 80 wt-%, at least about 90 wt-%, at least about 95 wt-%, or at least about 99 wt-%, of the total weight of the synthetic ELP (i.e., the combined molecular weight of these functional oligopeptide blocks may represent these percentages of the total molecular weight of the synthetic ELP).

Particular synthetic ELPs of interest have the sequences [(IPAVG)₄[(VPGVG)₂(VPGIG)(VPGVG)₂]₄(GAGAGS)₄]₃(SEQ ID NO:11, also referred to herein as “EPR011”) and [(IPAVG)₄[(VPGVG)₂(VPGIG)(VPGVG)₂]₂VTKHLNQISQSY[(VPGVG)₂(VPGIG)(VPGVG)₂]₂(GAGAGS)₄]₃(SEQ ID NO:12, also referred to herein as “EPR018”). These and other synthetic ELPs having ends as defined above include those comprising, or consisting of, the sequence (IPAVG, SEQ ID NO:2)ₓ(VPGXG, SEQ ID NO:1)_{y}(GAGAGS, SEQ ID NO:3)_{z}, wherein x, y, and z independently represent positive integers, such as in the case of each of x, y, and z independently representing positive integers within ranges selected from those from about 1 to about 250, from about 1 to about 100, and from about 1 to about 50. Yet further synthetic ELPs having ends as defined above include those comprising, or consisting of, [(IPAVG, SEQ ID NO:2)ₓ(GAGAGS, SEQ ID NO:3)_{y}](VPGVG, SEQ ID NO:8)_{z}[(IPAVG, SEQ ID NO:2)ₘ(GAGAGS, SEQ ID NO:3)ₙ], wherein m, n, x, y, and z independently represent positive integers, such as in the case of each of m, n, x, y, and z independently representing positive integers within ranges selected from those from about 1 to about 250, from about 1 to about 100, and from about 1 to about 50.

As in the case of synthetic biopolymers generally, synthetic ELPs as described herein, by virtue of their functional oligonucleotide blocks and/or by the use of a post-synthesis treatment as described herein, may be engineered or configured to achieve desired gelation characteristics, which may generally include the ability of the synthetic biopolymer (e.g., synthetic ELP) to undergo gelation, following heating of a solution of the synthetic biopolymer (e.g., synthetic ELP) at a sub-ambient temperature (e.g., 4° C.) or ambient temperature (e.g., 20° C.) to physiological temperature (e.g., 37° C.). Particular gelation characteristics that a synthetic biopolymer (e.g., synthetic ELP) may be engineered to achieve, as described herein, include a desired temperature at which the onset of gelation occurs (e.g., according to adaptations of its primary structure) and/or desired gelation kinetics (e.g., according to adaptations of its secondary structure).

According to particular embodiments, the gelation (or gelation characteristics) of a given synthetic biopolymer (e.g., synthetic ELP) as described herein may be more concretely defined according to a protocol in which the solution is an aqueous solution comprising 150 mg/ml of the synthetic biopolymer (e.g., synthetic ELP), the sub-ambient temperature is 4° C., and the physiological temperature is 37° C. In this case, the gelation (obtained by the synthetic biopolymer) is defined by rheological properties of a gel form of the synthetic biopolymer (e.g., the synthetic ELP), obtained after the heating of the aqueous solution at 4° C., with such heating consisting of a heating rate of 1° C. per minute and a holding period at 37° C. of 4 hours. The rheological properties include a gel storage modulus (G′) exceeding a gel loss modulus (G″), with such moduli being measured, for example, e.g., in pascals (Pa). In preferred embodiments, G′ exceeds G″ by at least 10%, at least 25%, at least 50%, or at least 100%. According to particular embodiments described herein in which gelation characteristics that are insufficient may be the basis for adjusting a post-synthesis treatment, insufficient gelation characteristics may include G′ exceeding G″ by less than a threshold percentage, such as any discreet value within the range of 20% to 200%. According to other particular embodiments described herein in which gelation characteristics that are excessive may be the basis for adjusting a post-synthesis treatment, excessive gelation characteristics may include G′ exceeding G″ by more than a threshold percentage, such as any discreet value within the range of 50% to 500%.

According to certain embodiments, the gelation (or gelation characteristics) of a given synthetic biopolymer (e.g., synthetic ELP) as described herein may be, more particularly, irreversible gelation (e.g., rheological properties that define gelation do not substantially return to their original values when the starting, lower temperature is restored). As in the case of gelation generally, irreversible gelation of a given synthetic biopolymer (e.g., synthetic ELP) as described herein may be more concretely defined according to a protocol in which the solution is an aqueous solution comprising 150 mg/ml of the synthetic biopolymer (e.g., synthetic ELP), the sub-ambient temperature is 4° C., and the physiological temperature is 37° C. In the case of irreversible gelation (obtained by the synthetic biopolymer), this may be defined by rheological properties of a temperature-cycled synthetic biopolymer (e.g., temperature-cycled synthetic ELP), obtained after subjecting the aqueous solution to a temperature cycle consisting of (i) heating of the aqueous solution at 4° C., with this heating consisting of a heating rate of PC per minute and a holding period at 37° C. of 30 minutes (e.g., to obtain a gel form of the synthetic biopolymer), followed by (ii) cooling from 37° C. to 4° C. at a cooling rate of 1° C. per minute and a holding period at 4° C. of 30 minutes. The rheological properties include a temperature-cycled storage modulus (TCG′) exceeding the initial aqueous solution storage modulus (IG′). In preferred embodiments, TCG′ exceeds IG′ by at least 10%, at least 25%, at least 50%, or at least 100%, meaning that the storage modulus, upon being subjected to this temperature cycle, does not return to its original value. Alternatively, but preferably in combination, the rheological properties may further include a temperature-cycled loss modulus (TCG″) exceeding the initial aqueous solution loss modulus (IG″), for example in the case of TCG″ exceeding IG″ by at least 10%, at least 25%, at least 50%, or at least 100%, meaning that the loss modulus, upon being subjected to this temperature cycle, does not return to its original value. According to particular embodiments described herein in which gelation characteristics that are insufficient may be the basis for adjusting a post-synthesis treatment, insufficient gelation characteristics (or more particularly insufficient irreversible gelation) may include TCG″ exceeding IG″ by less than a threshold percentage, such as any discreet value within the range of 20% to 200%. According to other particular embodiments described herein in which gelation characteristics that are excessive may be the basis for adjusting a post-synthesis treatment, excessive gelation characteristics may include TCG″ exceeding IG″ by more than a threshold percentage, such as any discreet value within the range of 50% to 500%.

According to other embodiments, irreversible gelation may be defined by successive increases in (or evolution of) G′ and/or G″ following each of a plurality (e.g., 2, 3, or 4) of temperature cycles as described herein, in which G′ and/or G″, obtained at the end of the holding period at 37° C. of 30 minutes, exceed its/their respective values obtained at an immediately-preceding temperature cycle, obtained at the end of the holding period at 37° C. of 30 minutes. For example, a second cycle storage modulus (2CG′) may exceed a first cycle storage modulus (1CG′) and/or a second cycle loss modulus (2CG″) may exceed a first cycle loss modulus (1CG″). In preferred embodiments, 2CG′ exceeds 1CG′ by at least 10%, at least 25%, at least 50%, or at least 100%, and/or 2CG″ exceeds 1CG″ by at least 10%, at least 25%, at least 50%, or at least 100%. Any of these differentials may likewise apply to the extent of a third cycle storage modulus (3CG′) exceeding a second cycle storage modulus (2CG′), a third cycle loss modulus (3CG″) exceeding a second cycle loss modulus (2CG″), a fourth cycle storage modulus (4CG′) exceeding a third cycle storage modulus (3CG′), a fourth cycle loss modulus (4CG″) exceeding a third cycle loss modulus (3CG″), etc. Threshold percentages by which a storage modulus of a given cycle may exceed that of a previous cycle, or by which a loss modulus of a given cycle may exceed that of a previous cycle, as the basis for gelation characteristics (or more particularly irreversible gelation) being insufficient or excessive may apply in an analogous manner as described above with respect to TCG″ exceeding IG″ by less than a threshold percentage (in the case of insufficient irreversible gelation) or by more than a threshold percentage (in the case of excessive irreversible gelation).

According to any of the protocols and associated rheological properties that define gelation, as described herein, the aqueous solution at 4° C. may have an initial aqueous solution storage modulus (IG′) substantially equal to (e.g., within about 10% of, or within about 5% of), or below, an initial aqueous solution loss modulus (IG″). According to any of the protocols and associated rheological properties that define gelation, as described herein, in some embodiments, the synthetic biopolymer would not have these associated rheological properties, absent post-synthesis treatment as described herein (e.g., according to embodiments in which post-synthesis treatment results in, or at least contributes to, advantageous gelation characteristics such as desirable structure and strength of the gelation). Rheological properties may be determined using apparatuses and their configurations and specifications, as well as any additional, specific conditions as described herein.

Further embodiments of the invention are directed to compositions comprising any synthetic biopolymer (e.g., synthetic ELP) as described herein. Representative compositions are suitable for injection and/or implantation in a human or animal body, such as in the case of a bone void filler composition. By being suitable for such use, the composition itself may be used for injection and/or implantation, or the composition may, prior to such use, be subjected to further processing, such as described herein, to provide an injectable or implantable composition (e.g., in the form received by the end user, such as a medical professional). Often, such injectable or implantable composition of a synthetic biopolymer (e.g., synthetic ELP), for example in the case of an injectable putty or an implantable sponge, may undergo final preparation steps performed by a medical professional (e.g., a surgeon), including hydration immediately prior to injection or implantation, optionally in combination with forming (e.g., to conform to a particular repair site within the body of a patient), such as in the case of implantation.

Accordingly, compositions that require only such final preparation steps, in some embodiments, may be referred to as hydratable compositions (e.g., a hydratable and injectable putty or a hydratable and implantable sponge). A putty or paste form of a hydratable composition may be pre-loaded into a syringe, and these putty or paste forms, as well as solid forms of hydratable compositions, may be more particularly in a freeze-dried (lyophilized) form. A particular solid form is a porous sponge comprising the synthetic biopolymer (e.g., synthetic ELP), after having been subjected to lyophilization, such as in the case of a lyophilized sponge. The porous sponge may optionally further comprise dispersed inorganic particles, such as particles of minerals as described herein (e.g., hydroxyapatite and/or calcium phosphate, such as β-tricalcium phosphate). These minerals may act as bone-mimicking materials, dispersed within a matrix formed by the porous sponge. Representative hydratable compositions may comprise all or substantially all, e.g., at least about 80 wt-%, at least about 90 wt-%, or at least about 95 wt-%, of (i) one or more synthetic biopolymers (e.g., synthetic ELPs) as described herein, or (ii) one or more synthetic biopolymers (e.g., synthetic ELPs) as described herein and mineral particles. In the case of (ii), exemplary hydratable compositions may comprise from about 10 wt-% to about 80 wt-%, such as from about 20 wt-% to about 45 wt-%, of synthetic biopolymer(s) and from about 30 wt-% to about 95 wt-%, such as from about 50 wt-% to about 85 wt-%, of mineral particles. In the case of (ii) the one or more synthetic biopolymer(s) in such hydratable compositions are preferably one or more synthetic ELPs having a polypeptide sequence comprising at least one biomineralizing block as described herein.

Hydration prior to or upon use (e.g., injection or implantation) is performed by contacting the hydratable composition with a suitable hydration fluid, such as sterile water, saline solution, whole blood, platelet rich plasma (PRP), or bone marrow aspirate. Following hydration, in view of the properties of the synthetic biopolymer as described herein, the final, hydrated composition is configured to undergo gelation (e.g., irreversible gelation) when introduced into the patient (e.g., at a defect or repair site). Such properties thereby result in the final composition having structural and/or mineral retaining characteristics that are particularly favorable for use in tissue repair.

In other embodiments, representative compositions are not necessarily injectable or implantable compositions, but can undergo further processing steps to provide such compositions. Generally, such compositions, according to embodiments of the invention, comprise a synthetic biopolymer (e.g., synthetic ELP) as described herein and may be in a solid form or an aqueous solution form. Either of these forms may have been subjected to post-synthesis treatment as described herein, for induction of β-sheet formation to a desired extent. For example, a solid form may have been subjected to freeze-drying (e.g., relatively “harsh” freeze-drying or relatively “mild” freeze-drying as described herein) and optionally water vapor annealing, washing with an organic liquid, or thermal exposure. An aqueous solution form may have been likewise subjected to one or more of these steps, as a post-synthesis treatment, and then further solubilized to provide such aqueous solution comprising the synthetic biopolymer, for example at a relatively high concentration (e.g., from about 100 mg/ml to about 300 mg/ml) for optional further processing as described herein, such as to provide an injectable or implantable composition. In other embodiments, an aqueous solution form may have been subjected, as a post-synthesis treatment, to thermal exposure as described herein, or a combination of freeze-drying and thermal exposure, with solubilization (e.g., to obtain a relatively high concentration as described above) occurring between these steps. Those skilled in the art and having knowledge of the present disclosure can appreciate that representative solid and aqueous solution forms may be obtained following any of a number of steps, including freeze-drying, water vapor annealing, washing with an organic liquid, and/or thermal exposure, which may be performed in any combination and any number of times with respect to a given, individual step (e.g., multiple cycles of freeze-drying with intermediate solubilization), for induction of β-sheet formation to a desired extent (e.g., as determined by FTIR or other properties indicative of an extent of physical cross-linking or β-sheet formation, as described herein).

Yet further embodiments of the invention are directed to methods for preparing compositions as described herein, comprising one or more synthetic biopolymers (e.g., one or more synthetic ELPs). Representative methods comprise: (a) separating the synthetic biopolymer(s) from a cell culture (e.g., according to a primary recovery) and thereafter performing one or more purification steps (e.g., warm centrifugation) to provide an initial synthesis composition comprising the synthetic biopolymer(s). Those skilled in the art having knowledge of the present disclosure will appreciate that the synthetic biopolymer(s), and the initial synthesis composition, will not necessarily have the desired gelation characteristics at this stage. In this regard, however, the methods may further comprise: (b) inducing β-sheet formation among molecules of the synthetic biopolymer(s) (e.g., synthetic ELPs), to provide a post-synthesis treated composition in a solid form or an aqueous solution form. According to preferred embodiments, step (b) is carried out to an extent that does not result in gelation. According to other embodiments, β-sheet formation may be induced at least partially during purification, such that step (b) may be performed simultaneously with step (a). For example, pre-drying thermal exposure as described herein may be used or manipulated as a post-synthesis treatment step (e.g., in the warm centrifugation) for at least partially, or possibly completely, inducing β-sheet formation, to the extent obtained in the synthetic biopolymer, such as following formulation or even prototype preparation.

Whether or not all or a portion of inducing β-sheet formation (using a post-synthesis treatment as described herein) occurs in step (a) above, the solid form or aqueous solution form provided in step (b) may include any of the particular forms described herein, having been subjected to such post-synthesis treatment, in order to “engineer” a limited extent of β-sheet formation that may, for example, increase polymer chain length and increase the content of β-sheets acting to template the formation of further β-sheet s. This may advantageously promote gelation and improve rheological/mechanical properties of a gel that is formed under given gelation conditions. In the case of an aqueous solution form being provided in step (b), this step may serve to increase its viscosity. In particular embodiments, step (b) may comprise freeze-drying, water vapor annealing, washing with an organic liquid, or a combination thereof, to provide the post-synthesis treated composition in the solid form. A subsequent step of solubilizing (e.g., the resulting freeze-dried and/or water vapor annealed and/or washed intermediate) may be used to provide the composition in the aqueous solution form. In other particular embodiments, step (b) may comprise freeze-drying, thermal exposure (e.g., pre-drying thermal exposure or post-drying thermal exposure), or a combination thereof, to provide the post-synthesis treated composition in the aqueous solution form, for example in the case of intermediate solubilization (e.g., following drying such as freeze-drying) occurring prior to post-drying thermal exposure. In general, although solubilization may be included at various points in a post-synthesis treatment for induction of β-sheet formation to a desired extent, solubilization alone typically does not contribute to this β-sheet formation. Solubilization, as needed to provide an aqueous form, either as an intermediate composition or the post-synthesis treated composition, may involve substantial solubilization as opposed to complete solubilization (e.g., complete solubilization may not be necessary in the practice of a given post-synthesis treatment).

Representative methods may optionally further comprise: (c) further processing the post-synthesis treated composition to provide an injectable or implantable composition of the synthetic biopolymer, with representative injectable or implantable compositions being those described herein. For example, this step may comprise, optionally following dispersing inorganic particles (e.g., particles of mineral as described herein) in the post-synthesis treated composition, preparing a mold of the post-synthesis treated composition (e.g., molding this composition into a desired shape), and freeze-drying the mold. The further processing, prior to such optional dispensing of inorganic particles and preparing a mold, may involve other preparation steps. Such preparation steps may include any of the post-synthesis treatment steps (i), (ii), (iii), and/or (iv) to influence gelation characteristics as described herein. Therefore, it can be appreciated that any of these steps can be performed between primary recovery of the synthetic biopolymer (e.g., synthetic ELP) and providing it as an injectable or implantable composition, with these steps inducing physical cross-linking, such as by β-sheet formation as described herein. According to a particular embodiment, further processing of the post-synthesis treated composition may be used to form an implantable composition, in the form of a freeze-dried (lyophilized) and hydratable sponge. Such sponge may be generally made, for example, by first forming an aqueous slurry comprising the one or more synthetic biopolymer(s) obtained as a post-treated composition as described herein (e.g., in solid form or aqueous solution form), and particles of mineral as described herein (e.g., mineral known to promote bone growth, such as hydroxyapatite, calcium phosphate, bioactive glasses, and/or bone particles), together with sufficient water, such as Milli-Q® (MQ) water, to form the slurry. A particular example of bone particles that can be utilized are granules of porcine anorganic bone matrix (pABM). The slurry is dispensed into a mold, which is then placed in a freeze-dryer to lyophilize the slurry and provide the sponge in the desired (molded) shape, such as a cylinder or disc. The sponge is then separated from the mold.

Optional further processing steps, subsequent to freeze-drying the mold, include milling (e.g., using a 2 mm screen) and filling of the resulting milled composition into syringes, such as to provide the injectable composition. Otherwise, such optional further steps may include machining of a solid composition, such as a sponge, to improve the final shape, for example in terms of smoothness. Whether or not such optional further steps are used, freeze-dried compositions, obtained from freeze-drying the mold, and typically following removal from the mold, may be subjected to sterilization, for example under gamma radiation with a dose of 25-40 kilo Gray (kGy) to provide the injectable or implantable composition, such as a hydratable, injectable putty or hydratable, implantable sponge.

Still further embodiments of the invention are directed to methods of treating a patient, comprising injecting or implanting a composition as described herein in the patient. This can result in the delivery of the composition to, and/or contacting of the composition with, a host bone or bone void of the patient or a tooth or tooth void of the patient, advantageously promoting bone or tooth repair, bone or tooth growth, and/or bone or tooth strengthening. The injected or implanted compositions have characteristics as described herein (e.g., structural and/or mineral retaining characteristics), which are particularly favorable for tissue repair.

### EXAMPLES

The following examples are set forth as representative of the present invention. These examples are not to be construed as limiting the scope of the invention as other equivalent embodiments will be apparent in view of the present disclosure and appended claims.

The synthetic elastin-like polypeptides, EPR011 and EPR018 were designed and synthesized. EPR011 has the sequence [(IPAVG)₄[(VPGVG)₂(VPGIG)(VPGVG)₂]₄(GAGAGS)₄]₃(SEQ ID NO: 11). It was formed by the following blocks: VPGVG (SEQ ID NO:8), a canonical ELP block inspired in the hydrophobic repetitive and temperature-responsive block found in human tropoelastin; VPGIG (SEQ ID NO:9), variation of the VPGVG (SEQ ID NO:8) block in which the 4^{th} amino acid is substituted by isoleucine, a more hydrophobic amino acid that aims at obtaining ELPs that coacervate at physiological conditions; IPAVG (SEQ ID NO:2), a plastic-like ELP block to enhance aggregation upon temperature increase; and GAGAGS (SEQ ID NO:3), a silk-like block that enables the formation of β-sheet crystalline structures in EPR011 gels that render those gels irreversible while increasing their mechanical strength. The expected molecular weight of EPR011 was 34.9 and the isoelectric point, pI, was 5.3. EPR018 has the sequence [(IPAVG)₄[(VPGVG)₂(VPGIG)(VPGVG)₂]₂VTKHLNQISQSY[(VPGVG)₂(VPGIG)(VPGVG)₂]₂(GAGAGS)₄]₃(SEQ ID NO:12). It was formed by the blocks as described above with respect to EPR011, but included the following, additional block: VTKHLNQISQSY (SEQ ID NO:7), a biomineralizing block with the ability to template the growth of hydroxyapatite for bone regeneration purposes. The expected molecular weight of EPR018 was 39.1 and the isoelectric point, pI, was 9.7.

### Strain Construction

Synthetic genes encoding EPR011 or EPR018 were ordered from a commercial supplier. The received plasmids were transformed into E. coli according to the supplier's protocol. The transformants were plated on 2*PY agar+antibiotic. Incubation was performed at 30° C. overnight. Afterwards, clones were selected and stored in glycerol stocks (50%) at −80° C. until further use.

### Fermentation and Protein Expression

According to steps performed in primary recovery, E. coli transformants expressing EPR011 or EP018 were inoculated in 2*PY+antibiotics (100 ng/μl neomycin) and incubated at 30° C. and 250 rpm overnight. The next day, 1/100 volume of preculture in 2*PY was inoculated for the fermentative production of EPR011 or EPR018 in 500 ml shake flasks with 100 ml of Terrific Broth (TB) medium+antibiotics (100 ng/μl neomycin). Cells were incubated at 37° C. and 250 rpm for ca. 3-4 hours. Once the optical density of the culture reached 0.6-0.8, the cells were induced with L-arabinose to a final concentration of 0.02% and incubated at 27° C. overnight. Cells were harvested the day after, using 50 ml conical tubes by centrifugation at 7186 relative centrifugal force (rcf) for 20 minutes at 4° C., and the obtained pellets were stored at −20° C. until further use, to enhance cell lysis due to the freezing and subsequent thawing, and to minimize protein degradation.

### Purification and Formulation of EPR011

The frozen pellets obtained from fermentation were thawed on ice. Once the material was completely thawed, the cells were resuspended in PBS and the pH was adjusted to 4.0. The suspension was then subjected to a heatshock step at 90° C. for 30 minutes to facilitate cell lysis and ELP release, as well as protease inactivation. Thereafter, the suspension was cooled down to 25° C., the pH was adjusted to 6.0, and 0.6 g/kg lysozyme (chicken egg white) was added to enhance ELP release. The liquid was slowly cooled down overnight to 4° C. under constant stirring. The resulting suspension was then centrifugated at 7186 rcf for 1 h at 4° C. to separate the ELP (suspended in the cold supernatant) from the residual biomass. The cold supernatant was separated and NaCl was added to it, to achieve a molarity of 1 M. According to a warm centrifugation step, the aqueous solution was placed in a water bath preheated at 35° C. for 45 min. This caused the coacervation of EPR011, which was separated from the liquid by centrifugation at 30° C. for 30 min at 7186 rcf. The hot supernatant was discarded, and the pellet (containing mostly EPR011) was re-suspended in half of the starting volume of cold Milli-Q® (MQ) water and maintained at 4° C. overnight under constant stirring to facilitate the resuspension of EPR011 in cold water. The following day, a cold centrifugation at 7186 rcf for 1 h at 4° C. was performed to further purify EPR011, which remained in the cold supernatant. The material was then analyzed by UPLC or SDS PAGE (described below) to confirm the purity. It can be appreciated that this purification cycle can be repeated if the purity is not satisfactory. According to these purification steps, an aqueous solution containing dissolved EPR011, based on protein content, was obtained.

This aqueous solution was subjected to a concentrating (and if needed a diafiltration) step using a 1 kDa regenerated cellulose membrane to remove any leftover salts and other small contaminants, possibly present in the suspension. The resulting liquid product was then added in a drop-wise manner to liquid N₂ to facilitate the formation of frozen EPR011 droplets/pellets (ca. 0.5 in diameter). This facilitated the freeze-drying of EPR011 by increasing the surface area of the frozen EPR011 and thereby promoting the removal of water by lyophilization. The obtained frozen droplets were placed in a Christ freeze-dryer with a slow drying program, and a final EPR011 dry product was obtained after 2 days. The purity was thereafter checked with Total Amino Acid (TAA) analysis.

### Purification and Formulation of EPR018

The synthetic ELP, EPR018, was purified in substantially the same manner as EPR011, although it was recognized that protease inactivation from the heatshock step was beneficial in terms of preventing degradation of EPR018 due to severing of the biomineralizing block in its polypeptide sequence. In addition, the cold supernatant, which was separated from the residual biomass, was adjusted to pH 10, prior to the NaCl addition.

### Preparation Method Overview

As in the case of EPR011, EPR018 at this point was recovered as a freeze-dried composition, following the same mild freeze-drying as described above for EPR011, which included freezing droplets in liquid N₂. The mild freeze-drying in each case was therefore part of the post-synthesis treatment. According to another procedure, this mild freeze-drying was replaced with harsh freeze-drying, and preparations using this procedure are designated “CFD” in the figures. In the case of harsh freeze-drying, 50 ml plastic tubes, each containing frozen 35 ml volumes of aqueous solution of EPR011 or EPR018 were placed in a freeze-dryer maintained at 0.05 millibar (mbar) absolute pressure with a condenser temperature of −80° C., and allowed to dry for 48 hours at room temperature. With respect to either of the freeze-drying procedures, steps involved in primary recovery, purification, and formulation, leading up to obtaining the freeze-dried compositions of EPR011 and EPR018, are labeled as steps 1-11 in the flow diagrams of FIG. 1A and FIG. 1B. Steps 1 and 2 in these figures can be considered steps providing an initial synthesis composition, prior to post-synthesis treatment.

Water Vapor Annealing, Washing with Ethanol, and Thermal Exposure

The freeze-dried compositions of EPR011 and EPR018, obtained as described above, were subjected to water vapor annealing using an isotemp vacuum oven (13 mbar) overnight (ca. 18 h) at room temperature to induce the formation of β-sheets from the GAGAGS (SEQ ID NO:3) blocks in the ELP sequence. Afterward, the water-annealed material was air-dried overnight at room temperature. Like the mild freeze-drying or harsh freeze-drying used to prepare the synthetic biopolymers, the water vapor annealing was also part of (i.e., a step in) the post-synthesis treatment. As an alternative to water vapor annealing, in other procedures, washing with ethanol was used. These different post-synthesis treatment steps, which were in specific examples compared as alternatives following freeze-drying, are labeled in step 12 in the flow diagram of FIG. 1A. According to FIG. 1B, however, solubilization followed by a step of thermal exposure, labeled as 12a/13a and 12b/13b, are used in place of either the water vapor annealing or washing with ethanol. As shown in this figure, thermal exposure can be an alternative post-synthesis treatment step for induction of β-sheet formation to a desired extent. According to the processes depicted in either FIG. 1A or FIG. 1B, starting with an aqueous solution as a particular form of a post-synthesis treated composition, namely the aqueous solution following freeze-drying and optional post-synthesis treatment steps, such as water vapor annealing or washing with an organic liquid (e.g., ethanol), as provided in step 13a/13b of FIG. 1A, or the aqueous solution following freeze-drying and thermal exposure provided in step 13a/13b of FIG. 1B, further processing of these compositions can be undertaken according to steps 14a-17a or 14b-16b as shown in these figures, prior to a sterilization step. For example, steps 14a-17a can represent further processing steps to provide an injectable putty or paste (e.g., as a hydratable composition), whereas steps 14b-16b can represent further processing steps to provide an implantable sponge (e.g., as a hydratable composition), comprising inorganic bone granules, namely porcine anorganic bone matrix (pABM), as shown in step 14b. FIG. 1C depicts a process with an alternative sequence of post-synthesis treatment steps that include freeze-drying in step 11 and washing with an organic liquid (in this case ethanol) in step 12. Subsequent drying at elevated temperature in step 13 can be considered part of the washing with organic liquid, or otherwise can be considered a separate post-synthesis treatment step of thermal exposure, to the extent that drying step 13 itself can be manipulated to influence gelation characteristics. According to the process depicted in FIG. 1C, an injectable composition without added inorganic bone granules can be prepared directly by filling the dried composition obtained from step 13 into syringes, according to step 14a, followed by sterilization at step 21. Otherwise, if inorganic bone granules (e.g., pABM) are to be added, according to step 17, then the intermediate preparation of a high viscosity composition may be desired, such as through steps of solubilization, followed by thermal exposure and subsequent cooling, according to steps 14b, 15, and 16. As described herein, insofar as the thermal exposure can influence gelation characteristics, this may manifest as an increase in solution viscosity at steps 15 and 16.

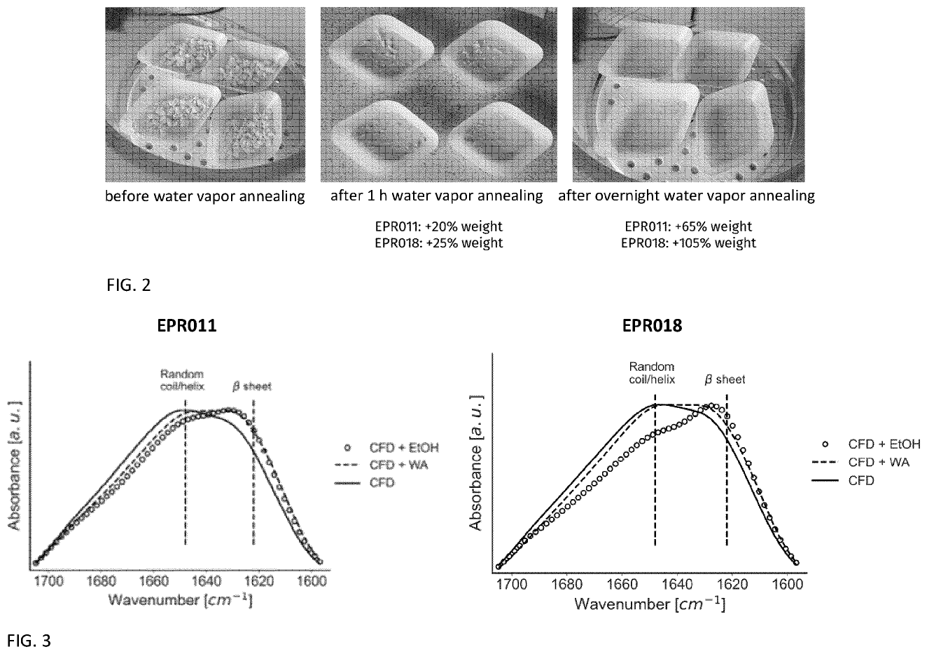

With respect to comparing the compositions prepared according to the processes shown in FIGS. 1A and 1B, the water vapor annealing step resulted in an observable change in morphology of both EPR011 and EPR018, as shown in FIG. 2. During the water vapor annealing step, the ELPs adsorbed a large amount of water (+65 wt-% for EPR011, +105 wt-% for EPR018), which resulted in a marked change in the secondary structure of the ELPs, as shown by FTIR. Likewise, this secondary structure was also found to be influenced by washing with ethanol. In this regard, FIG. 3 depicts FTIR scans of aqueous solutions of EPR011 and EPR018, following post-synthesis treatments of (i) harsh freeze-drying alone (“CFD”), (ii) harsh freeze-drying+water vapor annealing (“CFD+WA”), and (iii) harsh freeze-drying+washing with ethanol (“CFD+EtOH”). Based on the data shown in this figure, the differing post-synthesis treatment steps could be used to achieve dramatic changes in secondary structure of the ELPs, in terms of ß-sheet formation.

### Characterization and Results

Total amino acid (TAA) analysis: TAA analysis was done according using Waters Accq Tag method after chemical hydrolysis.

Ultra-High Performance Liquid Chromatography (UPLC): Detection of ELPs by UPLC was performed in a Waters HClass-Bio UPLC system (LC905) with a URP-UV 220 nm detector and using trifluoroacetic acid (TFA) as an ion paring agent. A Waters RP C4 column (1.7 μm, 50×2.1 mm) was used. The mobile phase A consisted of 100% MQ water and 0.05% TFA, while the mobile phase B consisted of 10% MQ water/90% acetonitrile plus 0.04% TFA. The system was operated at a flow rate of 0.4 ml/min and a column temperature of 20° C.

Thermogravimetric analysis (TGA): Thermogravimetric analysis (TGA) was performed on a Mettler Toledo DSC/TGA. Approximately 5 mg of sample was weighed into a pre-weighed aluminum oxide cup of 70 μl. The temperature program profile consisted of the following steps: Step 1, heating from 25 to 100° C. at a heating rate of 5° C./minute; Step 2, holding at 100° C. for 10 minutes; Step 3, heating from 100 to 200° C. at a heating rate of 5° C./minute; Step 4, holding at 200° C. for 10 minutes; Step 5, heating from 200 to 1000° C. at a heating rate of 40° C./minute; Step 6, holding at 1000° C. for 5 minutes; Step 7, cooling from 1000 to 25° C. at a cooling rate of 40° C./minute; Step 8, holding at 25° C. for 10 minutes. Moisture content was determined by the weight loss at 100° C. (step 2), and ash content was determined by the weight loss at 25° C. (step 8).

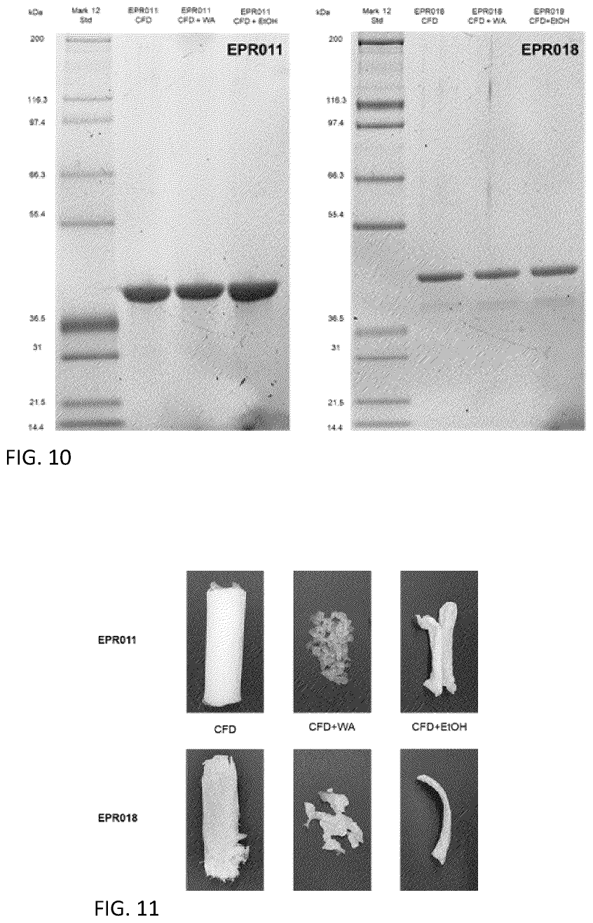

SDSPAGE: Sodium dodecyl sulphate-polyacrylamide gel electrophoresis (SDS-PAGE) was used to assess purity of synthetic biopolymers using NuPAGE 4-12% Bis-Tris gels from Invitrogen and following the protocol from the manufacturer. Each SDSPAGE sample was incubated at 70° C. for 10 minutes before analysis. Two gels were run using MOPS solution, for 50 min with 200 V, and Mark 12™ was used as the protein ladder. The resulting gel was stained with Sypro Red, using 30 ml of acetic acid (7.5 wt-%) and 6 μl of Sypro Red staining agent. FIG. 10 shows SDS-PAGE gels obtained for EPR011 and EPR018, in addition to these synthetic biopolymers following post-synthesis treatments of (i) harsh freeze-drying alone (“CFD”), (ii) harsh freeze-drying+water vapor annealing (“CFD+WA”), and (iii) harsh freeze-drying+washing with ethanol (“CFD+EtOH”), as described above. Advantageously, the different ways in which these polypeptides were processed did not materially affect their purities or molecular weights. This is despite the fact that the post-synthesis treatments (i), (ii), and (iii) resulted in differences in physical appearance of the dried materials, as shown in FIG. 11.

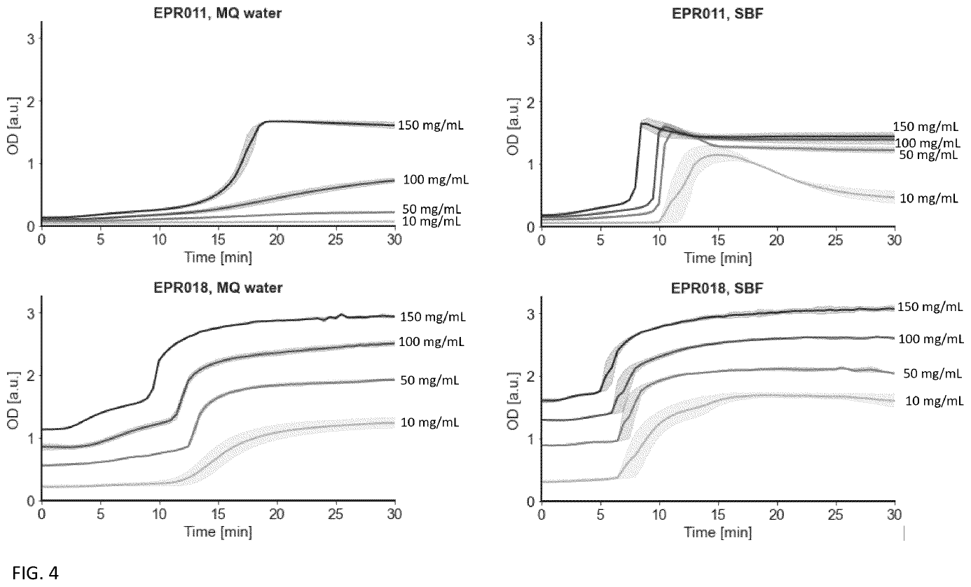

Optical density (OD): Optical density measurements were performed using aqueous ELP solutions at varying polypeptide concentrations (10, 50, 100, and 150 mg/ml) and in two different solvents: MQ water and simulated body fluid (SBF). In each case, 0.1 ml samples of aqueous solution were loaded onto 96-well plates, and the plates were then introduced to a photospectrometer preheated at 37° C. The changes in optical density, or turbidity, were recorded over time. These changes for aqueous solutions of EPR011 and EPR018 are shown in FIG. 4. Based on the graphs in this figure, the increases in turbidity occur over a shorter time period for EPR018 than for EPR011. Also, higher concentration for a given ELP directionally led to faster changes in turbidity.

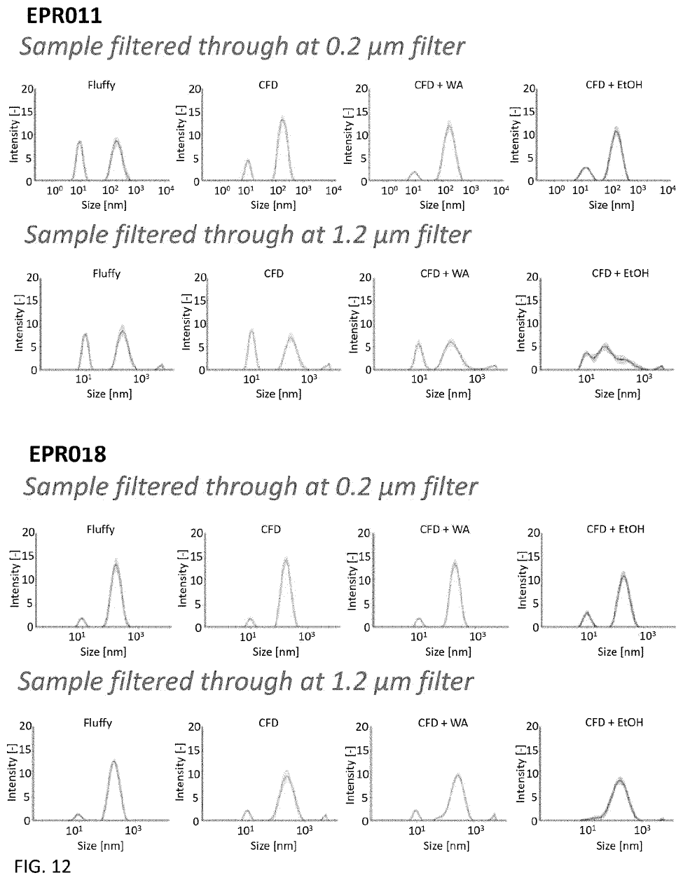

Dynamic Light Scattering (DLS): The particle size distribution of 0.5 mg/ml solutions of synthetic biopolymers was measured using a Zetasizer Nano Series dynamic light scattering (DLS) instrument (Malvern Instruments). Samples were dissolved in water and incubated at 4° C. for 1 hour. Subsequently, they were filtered using a 0.2 μm or 1.2 μm syringe filter, prior to analysis. Measurements were performed in plastic PS cuvettes (BrandTech Scientific) at 25° C. The laser power was adjusted automatically by the built-in auto-attenuation capability for each sample to an optimized range of counts. The acquisition time for each data point was 10 seconds, and 5 replicas were acquired per sample. FIG. 12 shows the DLS results obtained for 0.5 mg/ml solutions EPR011 and EPR018, in addition to these synthetic biopolymers following post-synthesis treatments of (i) harsh freeze-drying alone (“CFD”), (ii) harsh freeze-drying+water vapor annealing (“CFD+WA”), and (iii) harsh freeze-drying+washing with ethanol (“CFD+EtOH”), as described above. This figure provides the particle size distributions using the two types of filters, 0.2 μm or 1.2 μm, at 25° C.

The DLS analysis allows for the assessment of how different post-synthesis treatments in formulation and/or prototype preparation, affected the particle sizes of the diluted, 0.5 mg/ml, solutions of EPR011 and EPR018. The results indicated that CFD+WA and CFD+EtOH treatments increased the content of particles with larger diameters, providing evidence that indicates that WA or EtOH, as post-synthesis treatment steps, formed physical cross-links between polypeptide chains, thereby increasing the effective chain length of the biopolymers in solution. This facilitated the formation of a percolated network upon gelation, which was a factor in ultimately providing robust and irreversible gels. Further supporting the proposed phenomena, and illustrated in FIG. 12, is the finding that denaturing with 6M guanidine hydrochloride disrupts β-sheet formation among polypeptide strands, leading to only a single peak in solution, corresponding to the hydrodynamic diameter of a single ELP molecule.

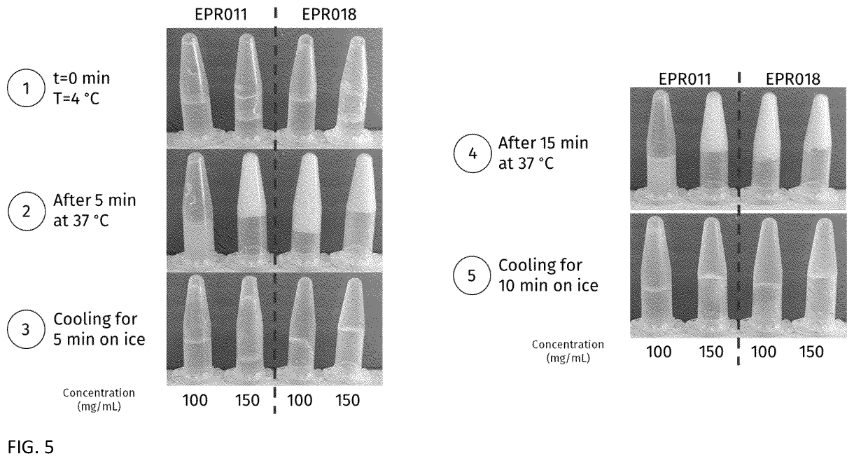

Gelation: Gelation was estimated through an inverted test tube technique. According to this procedure, aqueous solution volumes of ca. 1 ml with ELP concentrations of 100 mg/ml and 150 mg/ml were tested. The aqueous solutions were placed in Eppendorf tubes of 1.5 ml in volume, and subjected to 37° C. for 15 minutes. For these tests, samples of EPR011 and EPR018 after water vapor annealing were used, because these ELPs, when subjected to the relatively “mild” freeze-drying alone and without water vapor annealing, did not lead to irreversible gelation in the time scales used for these experiments. The results in FIG. 5 show the time evolution of the irreversibility of these ELPs after incubation. The water vapor annealed EPR018 formed irreversible gels after 5 min at 37° C. Those gels were able to maintain their shape, even after cooling on ice. The water vapor annealed EPR011 needed slightly more time, namely 15 min at 37° C., to form irreversible gels.

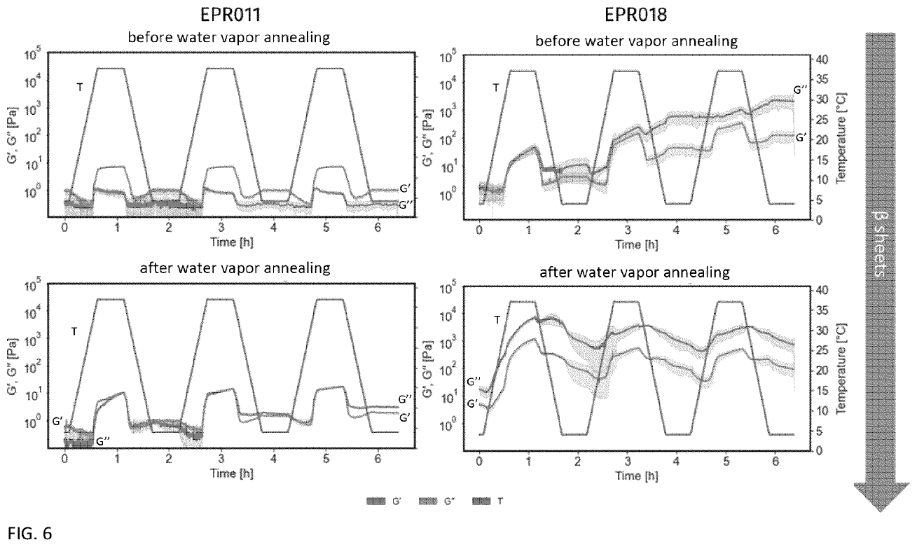

Shear rheology: Rheological characterization was performed to assess the ability of solutions of synthetic biopolymers to form hydrogels, and to characterize their viscoelastic properties. More specifically, the linear viscoelastic moduli of aqueous solutions containing EPR011 or EPR018, both before and after water vapor annealing that followed freeze-drying procedures (mild or harsh) as described above, were measured by small amplitude oscillatory shear rheology on a stress-controlled rheometer (Anton Paar MCR 301), equipped with a cone-plate geometry having a diameter of 50 mm, and cone angle of 1°. The experiments were performed at temperatures between 4 and 37° C., set by a Peltier system. Aqueous ELP solutions of 590 μl having 150 mg/ml (15 wt-%) polypeptide concentration were loaded onto the bottom plate with a pipette and at a temperature of 4° C., and these solutions were allowed to thermally equilibrate for 5-10 minutes. Low viscosity mineral oil (Sigma Aldrich) was applied to air-sample interfaces around the measuring geometry to prevent water evaporation. Rheological properties (storage modulus, loss modulus, and phase angle) were determined by applying an oscillatory shear strain at an oscillation frequency of 1 Hz, and a small strain amplitude of 0.3%. The temperature was increased from 4 to 37° C. at a rate of 1° C./minute, and held at 37° C. for 30 minutes, before bringing the temperature back again to 4° C. at a rate of 1° C./min, and maintaining 4° C. for an additional 30 minutes. This temperature cycle was repeated 3 times in total. The data in FIG. 6 show the evolution of the storage modulus (G′) and loss modulus (G″) of gels made from the aqueous solutions of EPR011 and EPR018, before and after water vapor annealing and following the same, mild freeze-drying procedure, over the temperature cycles. Based on this data, the water vapor annealing step impacted the ability of ELPs to form irreversible gels. In the case of EPR011, aqueous solutions of this polypeptide could not form a structured gel without water vapor annealing, i.e., only following this post-synthesis treatment step was it able to do so. Also, the gels became stiffer with every temperature cycle. For EPR018, the water vapor annealing step did not seem to provide any significant advantage, in terms of mechanical properties of the irreversible gel. However, this post-synthesis treatment step did clearly accelerate the formation of an irreversible gel, which attained much higher mechanical strength following the first temperature cycle after water vapor annealing.

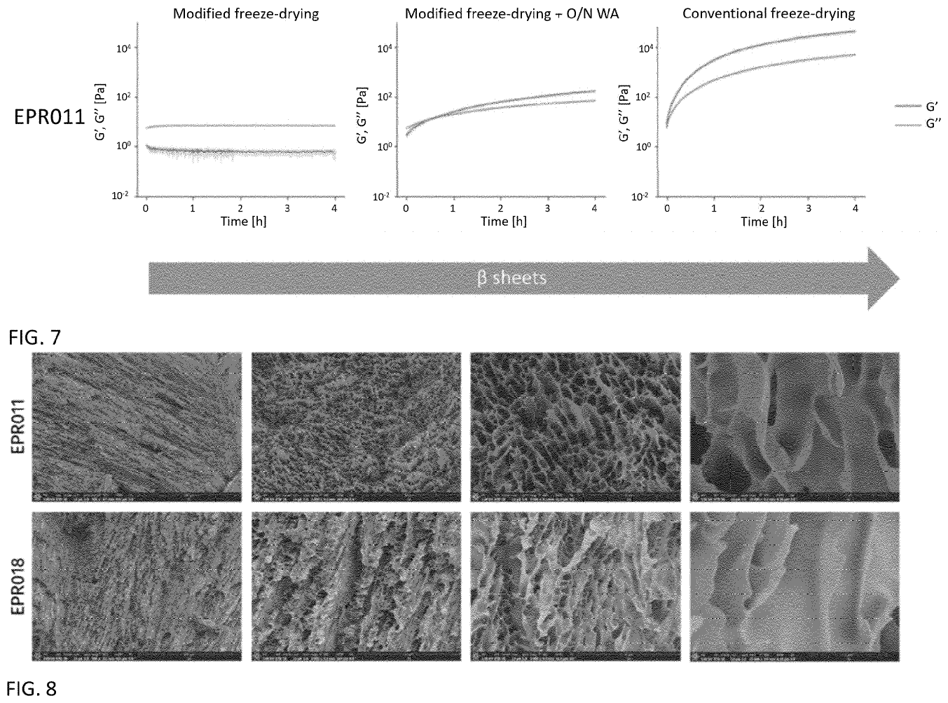

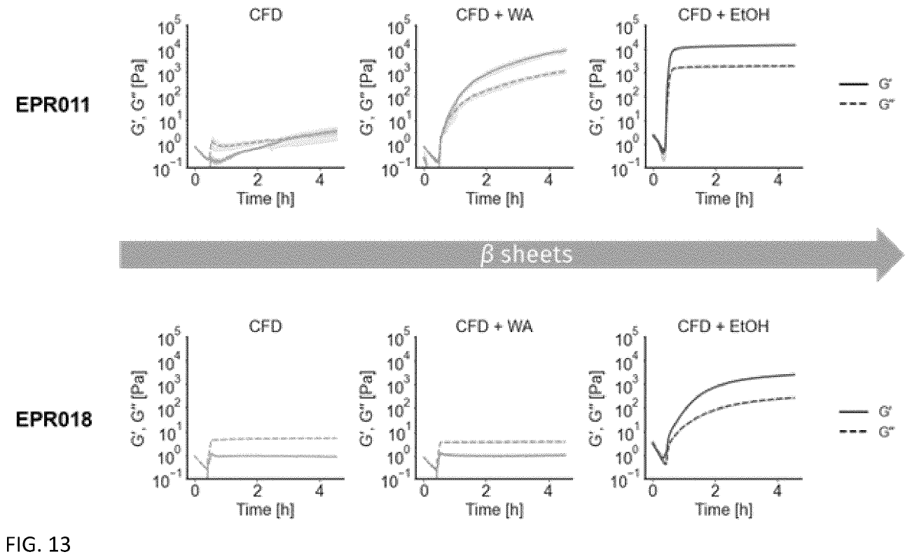

In other experiments, a “time sweep” temperature profile was used, according to which the temperature was increased from 4 to 37° C. at a rate of 1° C./minute, and held at 37° C. for 4 hours. The data in FIG. 7 show the changes over time for the storage modulus (G′) and loss modulus (G″) obtained for aqueous solutions of EPR011, following post-synthesis treatments of (i) mild freeze-drying alone (“Modified freeze-drying”), (ii) mild freeze-drying+water vapor annealing (“Modified freeze-drying+O/N WA), and (iii) harsh freeze-drying alone (“Conventional freeze-drying”). The same rheological properties over time were also measured for aqueous solutions of EPR011 and EPR018, following post-synthesis treatments of (i) harsh freeze-drying alone (“CFD”), (ii) harsh freeze-drying+water vapor annealing (“CFD+WA”), and (iii) harsh freeze-drying+washing with ethanol (“CFD+EtOH”), as described above, and the results are shown in FIG. 13. Based on the data from FIGS. 7 and 13, the formation of a stable gel, which can be indicated by G′ exceeding G″ upon heating, could be achieved using various post-synthesis treatments as described herein, although satisfactory gel formation does not necessarily result in all cases (e.g., in certain cases of using a mild freeze-drying procedure alone). As is evidenced by the results shown in FIGS. 3, 12, and 13, the effect of differing post-synthesis treatments, involving different steps following freeze-drying, can be determined based on a number of properties. Differing extents of β-sheet formation or content, are shown by FTIR, and these are further apparent from larger diameters obtained from cross-linking of synthetic biopolymers, shown by DLS. The FTIR and DLS results support a finding that physical cross-linking, at least partly resulting from β-sheet formation, influences the ability of synthetic biopolymers described herein to form gels, in addition to the mechanical properties of those gels. Importantly, the results demonstrate that CFD+WA and CFD+EtOH can be used in a post-synthesis treatment to form a desired degree of β-sheets in a given synthetic biopolymer, and even to tailor such biopolymer to achieve required gelation characteristics.

Scanning Electron Microscopy (SEM): ELP samples (in the form of films or hydrogels) were coated with gold using a vacuum coater, before imaging with an SEM.

Raman spectroscopy: The ELP samples were measured using a Renishaw inVia Raman microscope equipped with a 523 nm laser at 100% power, using an objective 50×LWD and an acquisition speed of 5×1 s.

Fourier Transform Infrared Spectroscopy (FTIR): Dry samples of synthetic biopolymers (in the form of films or hydrogels) were analyzed by FTIR to assess their secondary structure. Infrared spectra were measured in a Bruker Vertex 70 Attenuated Total Reflectance FTIR device equipped with a Harrick split pea accessory. For each measurement, 64 scans with a resolution of 2 cm⁻¹ were coded in the range of 650 to 4000 cm⁻¹. The secondary structure of the polypeptides is related to the C═O stretching vibration and can be determined by performing peak deconvolution over the amide I region (1595-1705 cm⁻¹). This was performed using the lmfit package for curve fitting from Python. The peak positions were allowed to shift 4 cm⁻¹ to obtain a reconstituted curve as close as possible to the original spectra. The amide I region from all spectra was normalized to its highest value, to facilitate the comparison between different samples. The Levenberg-Marquardt least-squares method was used for fitting, and a Gaussian model was selected for the band shape.

Secondary structure characterization of ELPs: Freeze-dried EPR011 and EPR018 samples were evaluated via FTIR spectroscopy to evaluate the secondary structure of these synthetic polypeptides before and after water vapor annealing. This was done to understand the effect of water vapor annealing on the conformation of the starting ELP material, prior to dissolution in water. From this data, it could be observed that water vapor annealing can add to the content of β-sheets formed after freeze-drying whether performed using a mild freeze-drying step or a harsh freeze-drying step.

Microstructure characterization of ELP hydrogels: Freeze-dried samples of certain ELP aqueous solutions that showed a gelling behavior in the shear rheology experiments were analyzed by SEM to investigate their microstructure. Initially, samples of these aqueous solutions, 80 μl in volume, were incubated in microcentrifuge tubes at 37° C. for 1 hour. The resulting hydrogels were submerged in liquid N₂ for 1 min, followed by lyophilization. The freeze-dried samples were then cryo-fractured and imaged via SEM. As shown in FIG. 8, these samples of EPR011 and EPR018 had an open porous structure, with no clear differences in porosity. These results indicated that the inclusion of a biomineralizing domain in EPR018 did not cause its microstructure to vary appreciably, compared to that of EPR011.

Biomineralization: After obtaining a detailed understanding of how post-synthesis treatment can affect, and can be used to control, the mechanical properties of synthetic biopolymers and their ability to form gels, these materials were further investigated for the biomineralization potential. The mineral of interest for this purpose was hydroxyapatite (HAP), the main inorganic component of bone. To induce mineralization, 50 μl of aqueous solutions of EPR011 or EPR018, at 100 mg/ml concentration, were used to cast films on circular plastic substrates and were left to dry in air. Subsequently, the films were subjected to ethanol treatment to render them insoluble and induce the formation of β-sheet s between the GAGAGS (SEQ ID NO:3) silk blocks, according to a process mimicking the effect of the water vapor annealing step. The films were subsequently dried in air and introduced into tubes containing either MQ water or 10 ml of a 1:1 solution of 12 mM NaH₂PO₄ (pH=7.4) and 20 mM CaCl₂. The films were incubated at 37° C. for one week. Thereafter, the films were rinsed twice with 10 ml of MQ water to remove soluble salts and left to dry overnight. The following conditions were evaluated in duplicate: (a) EPR011 film in MQ water, (b) EPR011 film in NaH₂PO₄+CaCl₂ solution, (c) EPR018 film in MQ water, and (d) EPR018 film in NaH₂PO₄+CaCl₂ solution. The presence of minerals was assessed using SEM, Raman spectroscopy, and FTIR.

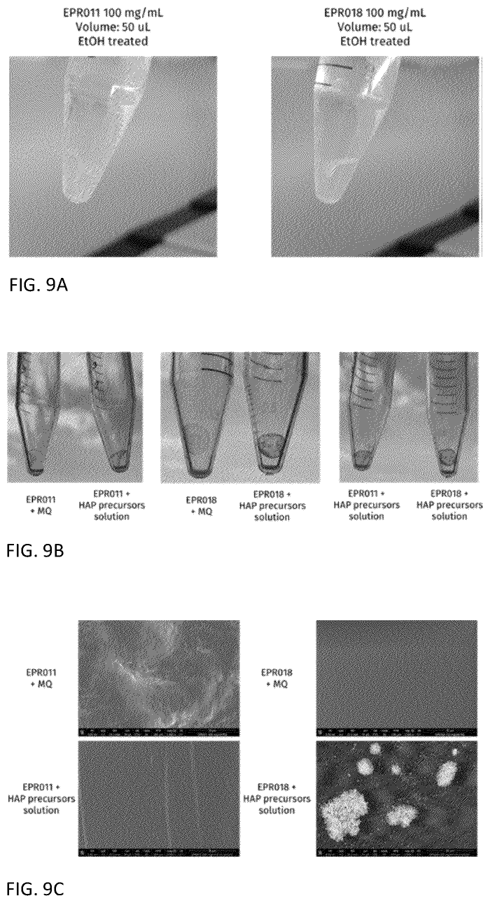

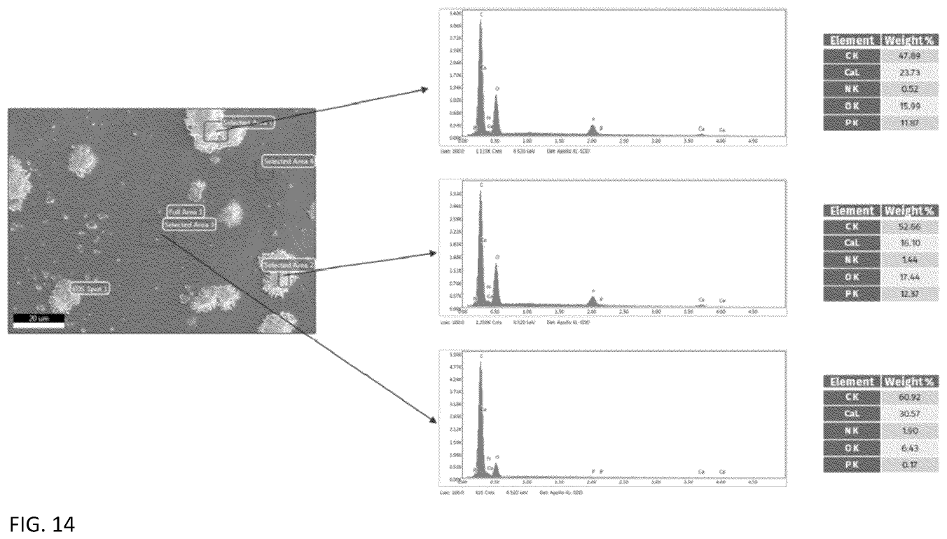

The films obtained during the biomineralization experiment are shown in FIG. 9A, and those obtained after this experiment are shown in FIG. 9B. The SEM images obtained for these films are shown in FIG. 9C. As indicated by these images, there was a clear visual difference between the EPR018 films incubated in the solution containing calcium and phosphate ions, and the rest of the films. This showed that only the EPR018 films in a solution containing calcium and phosphate ions were able to form mineral structures. This was confirmed by energy dispersive spectroscopy (EDS) imaging to identify crystal structures on the film surfaces, which structures were mostly formed by calcium and phosphate, the main components of HAP. The EDS images are provided in FIG. 14 (HAP=hydroxyapatite). Moreover, IR-ATR of EPR011 and EPR018 films showed that only the latter resulted in a small additional peak at around 1030 cm⁻¹, which is the position where hydroxyapatite would be expected. Features indicative of hydroxyapatite, namely a spectrum for the identified structures on the EPR018 films compatible with that of HPS, were likewise confirmed using Raman spectroscopy. These features were not detected on the EPR011 films. The biomineralization results were very encouraging, in terms of demonstrating the potential for synthetic biopolymers, and particularly those having one or more biomineralizing blocks as described herein, to drive the formation of mineral structures in ELP-based materials.

### Animal Study Procedures

Injectable Materials using EPR011 and EPR018, according to Examples 1 and 2, were prepared as follows: 50 ml tubes were filled with about 500 mg of ELP obtained according to the preparation methods described above, including a mild freeze-drying procedure. A 40 ml quantity of ethanol was added to each tube. The tubes were placed on a shaking table at room temperature for from 60 to 75 minutes, and the material was then centrifuged for 2 minutes. Ethanol was then substantially removed from the tube via manual pipetting. The material was then placed overnight in an oven at 50° C. under vacuum. The material, in the form of pellets, is placed into a syringe and capped.

Cylindrical plugs using EPR011 and EPR018, according to Examples 3 and 4, with diameter 5 mm and height 8 mm were prepared as follows: 50 ml tubes were filled with about 500 mg of ELP obtained according to the preparation methods described above, including a mild freeze-drying procedure. A 40 ml quantity of ethanol was added to each tube. The tubes were placed on a shaking table at room temperature for from 60 to 75 minutes, and the material was then centrifuged for 2 minutes. Ethanol was then substantially removed from the tube via manual pipetting. The material was then placed overnight in an oven at 50° C. under vacuum. The ELP was then dissolved in chilled PBS at 250 mg/ml, vortexed and centrifuged at 2600 rpm for 1 minute, and then visually inspected to determine whether all ELP had dissolved. The chilled temperature of the solution was maintained by chilling in an ice bath, as necessary. If not fully dissolved, the ELP was again vortexed and centrifuged for another minute, repeating until fully dissolved. The solution was then chilled for 10 minutes, followed by incubation at 80° C. for 1.5 minutes for EPR011 and 25 minutes for EPR018. Upon removal from the incubation, the material was stiff and chilled for 15 minutes in an ice bath to soften it. The softened material was then mixed sufficiently with porcine anorganic bone matrix (pABM—part number 30070 from DSM Biomedical, Inc., Exton, PA) and filled into pre-warmed (37° C.) molds, with caution to eliminate air bubbles. The molds were then sealed in a plastic bag with a damp wipe to maintain a humid environment and incubated at 37° C. for 4 hours. The mold tray was then removed from the plastic bag, lyophilized overnight, and the lyophilized samples were removed from the tray.

A control, according to Comparative Example 1 (CE1), was prepared by forming plugs of the same dimensions by lyophilizing a 4 wt % solids aqueous slurry comprising pABM, native fibrous collagen (DSM Biomedical, Inc. Part No. 4693-01), and soluble collagen (DSM Biomedical Inc., Part No. 6323-01). The weight ratio of pABM:fibrous collagen:soluble collagen was 90:5:5. The pABM specifications were as follows:

| Property | Value | Test Method |
|---|---|---|
| Particle Size | 0.25 to 1.00 mm | Particle Size Analysis |
| Distribution | | via Sieving |
| Mean Particle Size | 0.562 mm | Particle Size Analysis |
| | | via Sieving |
| % Porosity | 64% | MicroCT |
| Mean Pore Diameter | 150.7 μm | MicroCT |
| Pore Size | 0-650 μm | Mercury Porosimetry |
| Pore Interconnectivity | 133/mm³ | MicroCT |
| Particle Chemical | 99.56% | XRD |
| Composition | Hydroxyapatite, | Total Carbonate |
| | 0.44% Carbonate | |
| Crystallinity | 100% | XRD |
| Elemental Analysis | Arsenic <1 ppm | ICP-OES |
| (trace elements | Cadmium <1 ppm | |
| | Mercury <1 ppm | |
| | Lead <1 ppm | |
| | Total heavy metals | |
| | <50 ppm | |

The formed materials had the following compositions:

| | | | Collagen (50:50 | | ELP/Collagen: |
|---|---|---|---|---|---|
| | EPR011 | EPR018 | fibrous: | pABM | pABM |
| Example | (mg) | (mg) | soluble) (mg) | (mg) | by weight |
| 1 | 50 | | | | 1:0 |
| 2 | | 50 | | | 1:0 |
| 3 | 50 | | | 60 | 5:6 |
| 4 | | 50 | | 60 | 5:6 |
| CE1 | | | 6.5 | 58.5 | 1:9 |

The plugs are implanted into rabbits (Oryctolagus cuniculus) as follows (n=10 for Examples 1-4, n=6 for CE1). The medial femoral epicondyle of both limbs is exposed using a small incision (1-3 cm) for visualization of the medial femoral epicondyle. A k-wire is placed approximately 8.0 mm deep into the medial epicondyle, perpendicular to the long axis of the femur immediately proximal to the origin of the medial collateral ligament. A 5 mm cannulated drill bit (Arthrex, Ref #AR-1405) is placed over the k-wire and drilled to a depth of approximately 8 mm and 5-6 mm diameter under saline irrigation. The defect is flushed with saline to remove any bone fragments. The same procedure is performed on the contralateral limb. Each medial epicondylar defect is filled with one implant. For Examples 3 and 4 and CE1, the implant is simply placed in the defect.

The preparations of Examples 1 and 2 are pre-operatively prepared and inserted as follows: For the preparation of Example 1, (1) Draw up 0.2 cc of cold (˜4° C.) sterile saline with empty 3 cc syringe, (2) Draw up an additional 0.3 cc of air into the saline syringe (the syringe should be at 0.5 cc), (3) Remove cap from syringe containing pellets, (4) Attach syringe with the sterile saline to connector and then to pellet syringe, (5) Inject selected sterile saline from the liquid syringe into the pellet syringe, (6) Shake the syringes for 30 seconds, (7) Remove the empty syringe, (8) Carefully remove air from the syringe and replace the empty syringe, (9) Shake the syringe with the mixture for 30 seconds, (10) Reciprocate the mixture through the syringes twice (four total passes), (11) Remove empty syringe, (12) Carefully remove air from the syringe and replace the cap, (13) Place on ice bath (or equivalent 4° C. environment) for a minimum of 15 minutes, (14) Remove the cap and attach the injection needle, (15) Inject the mixture starting from the bottom of the defect and slowly move up until the defect is completely filled, and (16) Allow the mixture to cure for 10 minutes before closing the defect site. For the preparation of Example 2, (1) Draw up 0.2 cc of cold (˜4° C.) sterile saline with empty 3 cc syringe, (2) Draw up an additional 0.3 cc of air into the saline syringe (the syringe should be at 0.5 cc), (3) Remove cap from syringe containing pellets, (4) Attach syringe with the sterile saline to connector and then to pellet syringe, (5) Inject selected sterile saline from the liquid syringe into the pellet syringe, (6) Remove the empty syringe, (7) Carefully remove air from the syringe and replace the cap, (8) Shake the syringe with the mixture for 30 seconds, (9) Place on ice bath (or equivalent 4° C. environment) for a minimum of 15 minutes, (10) Remove the cap and attach the injection needle, (11) Inject the mixture starting from the bottom of the defect and slowly move up until the defect is completely filled, and (12) Allow the mixture to cure for 10 minutes before closing the defect site.

Once the sample is placed within the defect, the site, the surrounding tissues and joint are not irrigated or flushed. Following sample placement and cure timing (Examples 1 and 2), the subcutaneous layer is closed in a continuous pattern with 4-0 absorbable suture, followed by closure of the intradermal layer with a 4-0 monofilament absorbable suture. Sterile tissue glue is used as necessary to assist with suture closure. The survival time points are 8 weeks (+/−4 days) post-surgery for twenty-three (23) rabbits and 12 weeks (+/−4 days) post-surgery for twenty-three (23) rabbits. The animals are euthanized and pre-dissection radiographs and post-dissection MicroCT analyses are conducted on each femur specimen.

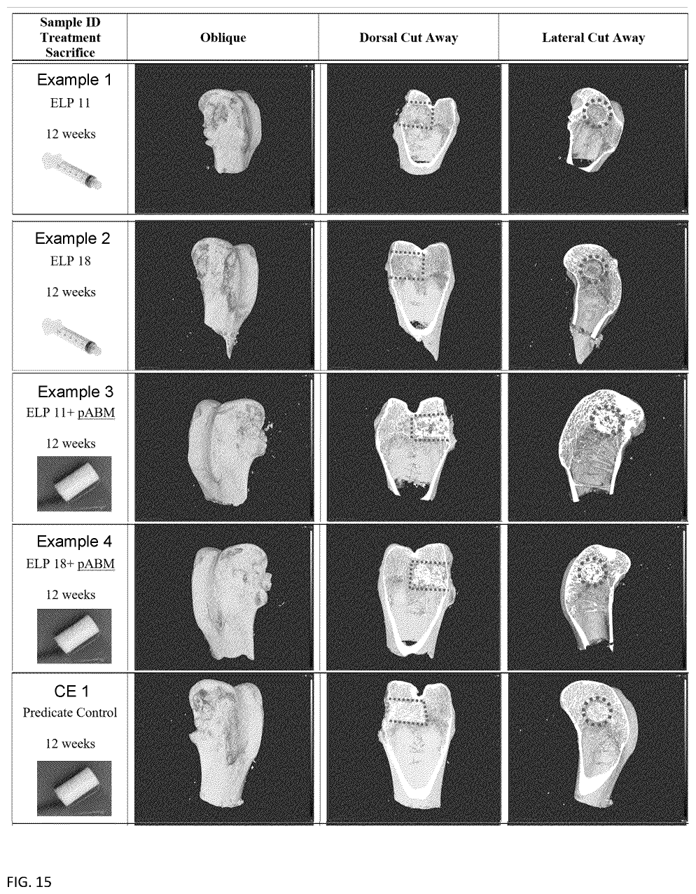

Post-dissection Micro CT scans are shown in FIG. 15. The defect site is highlighted using dots for the dorsal cut away and lateral cut away images. Each of Examples 1-4 and CE1 show radiopacity at the defect site. Cortical bone shows up radiopaque (white), while cancellous bone shows up as radiolucent (grayish). The defect site appears whiter in Examples 3 and 4 and CE1 because of the pABM present in those implants. In contrast, the injectable materials of Examples 1 and 2 do not contain added mineral content (pABM) and thus the white areas are not visible. Mineralization clearly occurred at each defect site. The similarity of the plugs of Example 3 and 4 to that of CE1 shows that the ELP itself is not detrimental in the plug form. Overall, no detrimental effects on the animal's bone structure, such as void enlargement, lesions around the void, or pocketing around the void, are observed.

A comparison of the injectable material of Example 1 with the plug of Example 3, and a comparison with the injectable material of Example 2 with the plug of Example 4, each indicate that cells are able to migrate through the gel formed in Examples 1 and 2 despite such gels being effectively nonporous relative to the porous plugs of Example 3 and 4. This is an encouraging result that evidences the ability of cells to migrate both through the formed gels of Examples 1 and 2 and the porous plugs of Examples 3 and 4. Generally, the mineralization would not be observed in Examples 1 and 2 unless cellular activity has occurred. The observed increase in radiopacity is indicative of bone healing.

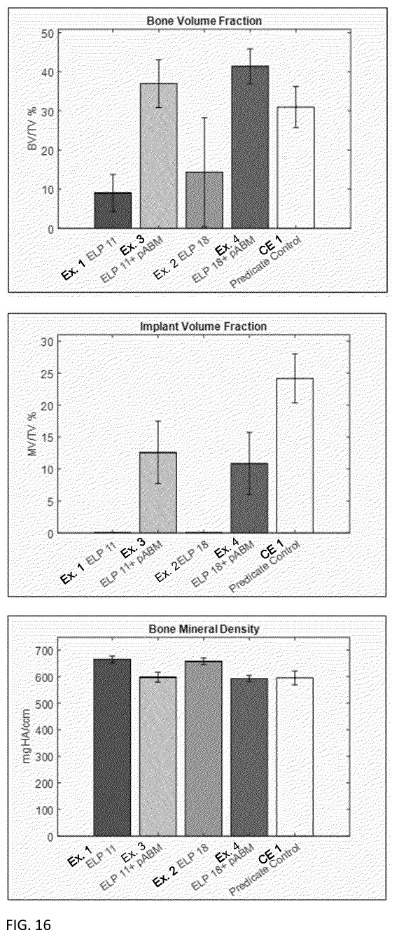

These results are consistent with the measurements shown in FIG. 16. Regarding Bone Volume Fraction, Examples 1 and 2 have less volume of mineral, so there is more room for new bone to grow in and replace the ELP at the 12-week timepoint. Implant volume fraction is also consistent with the mineral volume of the device, as Example 3 and 4 contain a ratio of ELP:pABM of 5:6 while CE1 contains a ratio of collagen:pABM of 1:9. The results of bone mineral density of new growth are representative of new tissue growing and replacing the ELP/collagen. Overall, the results indicate the potential for certain ELPs to be used in place of collagen to treat tissue defects.

Overall, aspects of the invention relate to compositions comprising synthetic biopolymers (e.g., synthetic ELPs) having desirable properties, for example by being suitable for use in implantable compositions to repair tissue, such as in bone void filler compositions. In some embodiments, the synthetic biopolymers can replace collagen used in conventional compositions. Those skilled in the art having knowledge of the present disclosure will recognize that various changes can be made to the disclosed synthetic biopolymers, compositions, methods of preparation, and methods of use, in attaining these and other advantages, without departing from the scope of the present invention. As such, it should be understood that the features of the disclosure are susceptible to modifications and/or substitutions, without departing from this scope. The specific embodiments illustrated and described herein are not limiting of the invention as set forth in the appended claims.

1. A synthetic biopolymer, wherein said synthetic biopolymer is engineered to undergo gelation, following heating of a solution of said synthetic biopolymer at sub-ambient (e.g., 4° C.) or ambient temperature to physiological temperature,
and undergoes physical cross-linking resulting from β-sheet formation among molecules of the synthetic biopolymer.

2. The synthetic biopolymer of claim 1, wherein said synthetic biopolymer is engineered by induction of at least a portion of said β-sheet formation.

3. The synthetic biopolymer of claim 1, wherein said synthetic biopolymer is engineered by (i) freeze-drying, (ii) water vapor annealing, (iii) washing with an organic liquid, (iv) thermal exposure or a combination thereof.

4. The synthetic biopolymer of claim 3, wherein said synthetic biopolymer is engineered by washing with the organic liquid, wherein the organic liquid comprises an alcohol, a hydrocarbon, an ether, a carboxylic acid, an ester, or a ketone.

5. The synthetic biopolymer of claim 4, wherein the organic liquid comprises an alcohol, wherein the alcohol is selected from the group consisting of methanol, ethanol, propanol, and butanol.

6. The synthetic biopolymer of claim 3, wherein said synthetic biopolymer is engineered by a combination of freeze-drying and thermal exposure, wherein said thermal exposure comprises one or both of (1) a pre-drying thermal exposure at a pre-drying temperature of at least about 35° C. and (2) a post-drying thermal exposure at a post-drying temperature of at least about 50° C.

7. A synthetic elastin-like polypeptide (ELP), having a polypeptide sequence comprising:
(a) one or more hydrophobic blocks of VPGXG (SEQ ID NO:1), wherein X represents any amino acid other than proline;
(b) one or more aggregation-enhancing blocks of IPAVG (SEQ ID NO:2), and
(c) one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6).

8. The synthetic ELP of claim 7, wherein X represents V or I.

9. The synthetic ELP of claim 7, wherein the polypeptide sequence further comprises:
(d) one or more biomineralizing blocks of VTKHLNQISQSY (SEQ ID NO:7).

10. The synthetic ELP of claim 7, having a molecular weight from about 15 kilo Daltons (kDa) to about 60 kDa.

11. The synthetic ELP of claim 7, wherein the polypeptide sequence consists of (a), (b), (c), and optionally
(d) one or more biomineralizing blocks of VTKHLNQISQSY (SEQ ID NO:7).

12. The synthetic ELP of claim 7, wherein a first end and/or a second end of the synthetic ELP is formed exclusively by (i) at least a portion of (b) the one or more aggregation-enhancing blocks of IPAVG (SEQ ID NO:2), and/or (ii) at least a portion of (c) the one or more β-sheet formation-inducing blocks of GAGAGS (SEQ ID NO:3), GAGAGY (SEQ ID NO:4), GAGYGA (SEQ ID NO:5), or GAGAGA (SEQ ID NO:6).

13. The synthetic ELP of claim 7, having the polypeptide sequence of:
(SEQ ID NO: 11)
[(IPAVG)₄[(VPGVG)₂(VPGIG)(VPGVG)₂]₄(GAGAGS)₄]₃,
or
(SEQ ID NO: 12)
[(IPAVG)₄[(VPGVG)₂(VPGIG)(VPGVG)₂]₂VTKHLNQISQSY
[(VPGVG)₂(VPGIG)(VPGVG)₂]₂(GAGAGS)₄]₃.

14. The synthetic ELP of claim 1, wherein said synthetic ELP is engineered to undergo gelation, following heating of a solution of said synthetic ELP at a sub-ambient or ambient temperature to a physiological temperature.

15. The synthetic biopolymer of claim 1,
wherein said solution is an aqueous solution comprising from 100 mg/mol to 300 mg/ml of said synthetic biopolymer or said synthetic ELP, said sub-ambient temperature is 4° C., and said physiological temperature is 37° C., and
wherein the gelation is defined by rheological properties of a gel form of the synthetic biopolymer or the synthetic ELP, obtained after said heating of said aqueous solution at 4° C., said heating consisting of a heating rate of 1° C. per minute and a holding period at 37° C. of 4 hours, said rheological properties including a gel storage modulus (G′) exceeding a gel loss modulus (G″).

16. The synthetic biopolymer of claim 1, wherein said gelation is irreversible gelation.

17. The synthetic biopolymer of claim 1, wherein said gelation is irreversible gelation, and
wherein said solution is an aqueous solution comprising 100-300 mg/ml of said synthetic biopolymer or said synthetic ELP, said sub-ambient temperature is 4° C., and said physiological temperature is 37° C., and
wherein said irreversible gelation is defined by rheological properties of a temperature-cycled synthetic biopolymer or a temperature-cycled synthetic ELP, obtained after subjecting said aqueous solution to a temperature cycle consisting of (i) said heating of said aqueous solution at 4° C., said heating consisting of a heating rate of 1° C. per minute and a holding period at 37° C. of 30 minutes, followed by (ii) cooling from 37° C. to 4° C. at a cooling rate of 1° C. per minute and a holding period at 4° C. of 30 minutes, said rheological properties including a temperature-cycled storage modulus (TCG′) exceeding an aqueous solution storage modulus (IG′).

18. A composition comprising the synthetic biopolymer of claim 1, wherein the composition is in a solid form or an aqueous solution form.

19. The composition of claim 18, wherein the solid form is a porous sponge comprising the synthetic biopolymer or the synthetic ELP following lyophilization, said porous sponge optionally further comprising dispersed inorganic solid particles.

20. A method for preparing a composition, the method comprising:
(a) separating the synthetic biopolymer of claim 1 from a cell culture to provide an initial synthesis composition, optionally following one or more purification steps;
(b) inducing β-sheet formation among molecules of the synthetic biopolymer or the synthetic ELP to provide a post-synthesis treated composition in a solid form or an aqueous solution form; and
(c) optionally further processing the post-synthesis treated composition to provide an injectable or implantable composition of the synthetic biopolymer or synthetic ELP.

21. The method of claim 20, wherein step (b) comprises (i) freeze-drying, (ii) water vapor annealing, (iii) washing with an organic liquid, (iv) thermal exposure or a combination thereof, and wherein the post-synthesis treated composition is provided in the solid form.

22. The method of claim 20, wherein step (b) comprises (i) freeze-drying, (ii) water vapor annealing, (iii) washing with an organic liquid, (iv) thermal exposure or a combination thereof, and wherein the method further comprises, prior to step (c), a step of solubilizing to provide the post-synthesis treated composition in the aqueous solution form.

23. The method of claim 21, wherein step (b) comprises washing with the organic liquid, wherein the organic liquid comprises an alcohol, a hydrocarbon, an ether, a carboxylic acid, an ester, or a ketone.

24. The method of claim 23, wherein the organic liquid comprises an alcohol, wherein the alcohol is selected from the group consisting of methanol, ethanol, propanol, and butanol.

25. The method of claim 21, wherein step (b) comprises a combination of freeze-drying and thermal exposure, wherein said thermal exposure comprises one or both of (1) a pre-drying thermal exposure at a pre-drying temperature of at least about 35° C. and (2) a post-drying thermal exposure at a post-drying temperature of at least about 50° C.

26. The method of claim 20, wherein step (c) comprises, optionally following dispersing inorganic particles in the post-synthesis treated composition, preparing a mold of the post-synthesis treated composition, and freeze-drying the mold.

27. A method of treating a patient, comprising implanting the composition of claim 18.
